(19) United States
(12) Patent Application Publication
(10) Pub. No.: US 2026/0327566 A1
(43) Pub. Date: Oct. 22, 2026

(54) POOLED MESENCHYMAL STROMAL CELL DERIVED CELLS, CELL- AND CELL-FREE PREPARATIONS THEREOF

(71) Applicants: DRK BLUTSPENDEDIENST BADEN-WÜRTTEMBERG-HESSEN GGMBH, Frankfurt am Main (DE); UNIVERSITÄT HEIDELBERG, Heidelberg (DE)

(72) Inventors: Karen BIEBACK, Speyer (DE); Helene WILLER, Weinheim (DE); Richard SCHÄFER, Buggingen (DE); Erhard SIEFRIED, Frankfurt am Main (DE); Gabriele SPOHN, Sulzbach am Main (DE); Kimberly MORGENROTH, Darmstadt (DE); Thomas SCHMITZ-RIXEN, Frankfurt am Main (DE)

(21) Appl. No.: 19/103,441

(22) PCT Filed: Aug. 16, 2023
(86) PCT No.: PCT/EP2023/072626

§ 371 Date: Feb. 12, 2025

(30) Foreign Application Priority Data
Aug. 16, 2022 (EP) 22190625.8

(51) Int. Cl. C12N 5/077; A61K 35/12; A61K 35/28; A61K 38/18; A61K 38/19; A61K 38/20

(52) U.S. Cl. CPC C12N 5/0669; A61K 35/28; A61K 38/1808; A61K 38/1825; A61K 38/1833; A61K 38/1841; A61K 38/185; A61K 38/1866; A61K 38/19; A61K 38/193; A61K 38/2006; A61K 38/204; A61K 2035/124

(57) ABSTRACT

The invention is based on a pooling strategy and cell passaging strategy for mesenchymal stromal cells (MSCs), which provide highly active master bank cell preparations and working bank cell preparations which can be directly used or, after freezing and thawing, cultured and expanded to yield either cell products or cell free products, such as cell-free culture supernatants, that are therapeutically active and can be used in various clinical applications. The invention provides methods for pooling MSCs, as well as the MSC preparations or cell-free compositions obtained from such MSC preparations.

### CROSS-REFERENCE TO RELATED APPLICATION

The present application is a National Stage Application of PCT International Application No.: PCT/EP2023/072626 filed on Aug. 16, 2023, which claims priority to European Patent Application 22190625.8, filed with the European Patent Office on Aug. 16, 2022, the disclosures of which are incorporated herein by reference in their entirety.

### FIELD OF THE INVENTION

The invention is based on a pooling strategy and cell passaging strategy for mesenchymal stromal cells (MSCs), which provide highly active master bank cell preparations and working bank cell preparations which can be directly used or, after freezing and thawing, cultured and expanded to yield either cell products or cell free products, such as cell-free culture supernatants, that are therapeutically active and can be used in various clinical applications. The invention provides methods for pooling MSCs, as well as the MSC preparations or cell-free compositions obtained from such MSC preparations.

### BACKGROUND

Human Mesenchymal Stromal Cells (hMSCs) have been widely investigated in cellular therapies. Specifically, MSCs exert therapeutic activities, most likely by secreting factors associated with chemoattraction, cell proliferation and differentiation, immunomodulation, angiogenesis, anti-apoptosis, anti-fibrosis, and even anti-microbial effects (1, 5-7). This has led to several promising preclinical studies, as well as phase I and II clinical trials. Yet, transition to phase III and IV clinical trials, or even marketing authorization, is remarkably slow. Next to safety and efficacy issues, often related to inconsistent study results, the so far tested hMSC therapies have been proven neither cost-effective, nor competitive against best-practice therapies (12). Next to technical obstacles (e.g. up-scaling and cryopreservation), issues pertaining to hMSC biology, such as donor variabilities, functional senescence, and the large variety of proposed mechanisms of action (MoAs), are increasing the complexity even further (13). Thus, to obtain robust and valid clinical data, it is of utmost importance to manufacture a substantial amount of hMSC doses from highly reproducible clinical hMSC products that can be tested in large randomized clinical trials. Furthermore, these products and their clinical evaluation require approval by the competent regulatory authorities. These expect a thorough scientific approach addressing GMP-compatible manufacturing, comprehensive quality control and in-depth preclinical efficacy and safety testing (13).

Upscaling issues of hMSCs were intensely discussed when a large phase III clinical trial failed to meet its clinical endpoint: the respective product “Prochymal™”, an allogeneic hMSC therapeutic, expanded in vitro to produce numerous clinical doses, lacked efficacy. In contrast, other allogeneic hMSC products, expanded to only few clinical doses, reproducibly showed efficacy in trials for steroid-refractory graft-versus-host-disease (GvHD) (14). This suggests that hMSCs manufacture should be carefully balanced to yield a sufficient number of clinical doses, but with only few population doublings during ex vivo production.

Inconsistent results from clinical trials may also result from donor-to-donor variability when hMSCs are manufactured from single donors (15). To address this, hMSC pooling concepts were developed. As one example, the product “MSC-FFM” was manufactured from pooled bone marrow (BM) mononuclear cells (MNCs), containing hMSC precursors as well as alloreactive immune cells of eight healthy 3rd-party donors (16). Besides reducing donor variability, an allogeneic immune reaction was intended to produce immunologically primed hMSCs with higher immunosuppressive strength. Indeed, for these cells beneficial effects in children and adults with severe steroid-refractory GvHD were reported (17, 18). Yet, highly immunosuppressive hMSCs may not be the first choice when aiming at chronic wound healing. Another example of a pooled hMSC product is “Stempeucel®”, successfully evaluated in critical limb ischemia. Here, a donor master cell bank (MCB) of single donor hMSCs after passage 1 was established (19). Next, a working cell bank (WCB) was generated by pooling hMSCs from three donors. This was then further expanded for five passages until the final product was cryopreserved. While the pooled “MSC-FFM” product requires establishing a new pooled hMSC MCB from scratch, the single-donor “Stempeucel®” hMSCs MCB concept allows high batch-to-batch consistency as recently shown (20). Yet, replicative aging within the five passages until reaching final product formulation may affect the quality of the clinical product (21).

In addition to donor-to-donor variance and extensive expansion, cryodamage and dosing issues are discussed to compromise the success of hMSCs clinical translation. To ease manufacturing and delivery, hMSCs are typically expanded ex vivo and then cryopreserved as clinical products, which are then shipped to the patient's bedside, where they are administered directly after thawing. Yet, cryopreservation may affect clinical potency associated with a heat-shock response, reduced immunomodulatory and homing capacity (27-29) and increased tissue factor expression (30).

Human Mesenchymal Stromal Cells (hMSCs) are a promising source for cell-based therapies. Yet, transition to phase III and IV clinical trials is remarkably slow. To mitigate donor variabilities and to obtain robust and valid clinical data, the inventors aimed first to develop a manufacturing concept balancing large-scale production of pooled hMSCs in a minimal expansion period, and second to test them for key manufacture and efficacy indicators in the clinically highly relevant indication such as wound healing.

### BRIEF DESCRIPTION OF THE INVENTION

In context of the present invention an exemplary, to which the invention is not generally restricted, novel clinical-scale manufacturing concept was developed which comprises six single donor hMSCs master cell banks that are pooled to a working cell bank from which an extrapolated number of 70,000 clinical doses of 1×10⁶ hMSCs/cm² wound size can be manufactured within only three passages. The pooled hMSC batches showed high stability of key manufacture indicators such as morphology, immune phenotype, proliferation, scratch wound healing, chemotactic migration and angiogenic support. Repeated topical hMSCs administration significantly accelerated the wound healing in a diabetic rat model by delivering a defined growth factor cargo (specifically BDNF, EGF, G-CSF, HGF, IL-1α, IL-6, LIF, osteopontin, VEGF-A, FGF-2, TGF-β, PGE-2 and IDO after priming) at the specific stages of wound repair, namely inflammation, proliferation and remodeling. Specifically, the hMSCs mediated epidermal and dermal maturation and collagen formation, improved vascularization, and promoted cell infiltration. Kinetic analyses revealed transient presence of hMSCs until day (d) 4, and the dynamic recruitment of macrophages infiltrating from the wound edges (d3) and basis (d9), eventually progressing to the apical wound on d11. In the wounds, the hMSCs mediated M2-like macrophage polarization starting at d4, peaking at d9 and then decreasing to d11.

This exemplary study of the invention establishes a standardized, scalable and pooled hMSC therapeutic, delivering a defined cargo of trophic factors, which is efficacious in diabetic wound healing by improving vascularization and dynamic recruitment of M2-like macrophages. This decision-making study now enables the validation of pooled hMSCs as treatment for impaired wound healing in large randomized clinical trials.

Therefore, and generally, and by way of brief description, the main aspects of the present invention can be described as follows:

In a first aspect, the invention pertains to a method for the production of a donor cell pool of expandable cells derived from bone marrow samples of multiple genetically non-identical bone marrow donors, the method comprising the steps of:
(a) Providing two or more bone marrow donor samples of genetically non-identical (or distinct) bone marrow donors,
(b) Separately passaging bone marrow derived cells from each bone marrow donor sample of (a) into a (separate) cell culture receptacle, such as a culture flask or dish (p0), bioreactor or 3D expansion system;
(c) Culturing the bone marrow derived cells of each donor separately;
(d) Separately passaging a portion of each culture of bone marrow derived cells from (c) into a new cell culture receptacle, such as a culture flaks or dish (p1);
(e) Culturing the bone marrow derived cells of each donor separately;
(f) Passaging and pooling (combining) a portion of each culture of bone marrow derived cells from (e) into one single cell culture receptacle to obtain the donor cell pool (p2).

In a second aspect, the invention pertains to a working cell bank and or a clinical product produced with the method of the invention.

In a third aspect, the invention pertains to a preparation of clinical grade pooled mesenchymal stromal cells (MSCs) produced with the method of the invention.

In a fourth aspect, the invention pertains to a preparation of clinical grade pooled mesenchymal stromal cells (MSCs) for use in the treatment of a disease or condition in a subject.

In a fifth aspect, the invention pertains to a method for manufacturing a therapeutically active composition, such as a cell-free growth factor containing composition the method comprising performing a method according to the invention, or providing a working call bank and/or the clinical product, or the preparation, of any aspect of the invention, and subsequently culturing and/or expanding the MSC material in a cell culture, and harvest a composition into which the MSC material secreted one or more cell factors, to obtain the therapeutically active composition.

In a sixth aspect, the invention pertains to a therapeutically active composition, containing one or more growth factors, produced with the method of the invention.

In a seventh aspect, the invention provides a pharmaceutical composition, comprising a cell or cell-free material of any aspect of the invention, or obtained by, or obtainable by, any aspect of the invention.

### DETAILED DESCRIPTION OF THE INVENTION

In the following, the elements of the invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

In a first aspect, the invention pertains to a method for the production of a donor cell pool of expandable cells derived from bone marrow samples of multiple genetically non-identical bone marrow donors, the method comprising the steps of:
(a) Providing two or more bone marrow donor samples of genetically non-identical (or distinct) bone marrow donors,
(b) Separately passaging bone marrow derived cells from each bone marrow donor sample of (a) into a (separate) cell culture receptacle, such as a culture flaks or dish (p0);
(c) Culturing the bone marrow derived cells of each donor separately;
(d) Separately passaging a portion of each culture of bone marrow derived cells from (c) into a new cell culture receptacle, such as a culture flaks or dish (p1);
(e) Culturing the bone marrow derived cells of each donor separately;
(f) Passaging and pooling (combining) a portion of each culture of bone marrow derived cells from (e) into one single cell culture receptacle obtain the donor cell pool (p2).

Methods steps are performed in sequential numberings order only insofar it is technically necessary. The scope of the present method claims shall not be understood to be restricted by the mere arbitrary choice of method step numbering. However, a preferred embodiment pertains to the exact indicated sequence of method steps.

The invention is in part predicated upon the observation that the pooling method of the invention structurally alters the MSC preparation in the following passages such, that the pooled cells in accordance to the invention produce, secrete and harbour various differential bioactive factors and activities. Some of those factors are shown in the example section herein below. In particular provided in context of the invention are therefore preparations of MSCs, either containing cells or being cell free, or cell-derived, such as lysed cells, or extracts, that are obtained from a culture of the MSC donor cell pool as described herein above. Hence, provided are a cellular MSC product produced according to the invention, a MSC in vitro (such as culture supernatant, Extracellular Versicles (EVs), Proteins, etc) derived a cellular product, as well as in-vivo released factors of the MSC preparations of the inventio, preparations containing factors present in the MSCs, as well as in-vivo induced factors produced from the MSC of the invention.

The term “genetically non-identical” shall refer not to absolute identity, but identity in the sense of genetics, which also can be described as “non-clonal” and which refers to a genetic heritage which is other than genetically “identical” twins, or inbred (clonal) animals, which are highly similar to genetic twins. The term shall not be understood to be a 100% identity, which would in view of spontaneously occurring single mutations or epigenetics would be nonsensical. The term “monogenic” when used in context to describe a sample or composition of cells refers to these cells originating from a common source or having the same (such as clonal) genetic background. The term “polygenic” on the other hand refers to a composition of cells originating from different sources and having different genetic backgrounds in the sense of genetically non-identical as defined above. In context of the present invention a “polygenic MSC preparation” is a composition comprising MSCs having distinct genetic backgrounds, for example MSCs which originate from at least two genetically distinct bone marrow donors.

The term “passaging” or “passage” shall refer to the transfer of all or a portion cells, either from an initial source such as a biological sample or from a previous cell culture, into a new cell culture. Preferably, the transfer is done into a new cell culture receptacle, such as a dish, bottle or flask, having fresh cell culture medium that allows for a further culturing and/or expansion of the cells. The passaging can comprise that certain fractions of cells are transferred such as only adherend cells, or soluble cells. In some embodiments, a passage may also include that non adherent cells are removed together with old cell culture medium, and new cell culture medium is added to the remaining adherent cells to further grow and expand the adherent cell fraction. For the purpose of the present invention passages are counted starting with p0, which is the transfer of the cells from a biological sample into a cell culture receptacle for further culturing for the first time. The follow up passages are counted in natural numbers p1, p2, . . . Pn. A passage is defined from the initial completed transfer to the moment the next transfer is started. Basically, as soon as a new receptacle or medium is provided a new passage begins.

In the current invention a cell culture passage line is started from p0 for a monogenic cell preparation, which stays monogenic until the first pooling occurs. It is preferred that the method of the invention at least involves P0 and P1 to be monogenic, and a combination (Pooling) of cell cultures is done at onset of P2. Whether the culturing of multiple monogenic cell cultures is done in parallel or sequentially, wherein a part of the cells are stored by freezing and thawed for pooling, is also covered by the present invention.

A “cell culture receptacle” shall be understood to pertain to any container suitable for cell culture and preferably suitable to grow and expand cells in or on a medium. Typical cell culture receptacles are flasks, plates, multi-well plates, bioreactors and the like.

As used herein the terms “bone marrow derived cells” (BMDCs) refer to a cell population derived from bone marrow samples that includes or comprises mesenchymal stem (or stromal) cells (MSCs).

In one embodiment, the method of the invention is further comprising a step (f′) subsequent to (f) freezing and storing the donor cell pool as a working cell bank.

In yet another embodiment, the method of the invention is further comprising a step (g) culturing the donor cell pool, either directly from step (f), or after thawing a cell sample of the working cell bank of the previous embodiment.

The term “cell material” as used in context of the present invention shall in particular pertain to materials comprising biological cell material, such as living or dead cells, preferably living cells, as well as any secreted factors derived from the culturing of such cells. In certain preferred embodiments, however, the present invention pertains to cell material comprising living biological cells. The expression “biological cell” in context of the invention shall preferably refer to a mammalian cell, most preferably a human cell. Certain embodiments of the invention pertain to cell-free compositions that do not comprise mesenchymal stromal cells, but for example are derived from such cells. In the present disclosure, the term “mesenchymal stromal cell” is a multipotent stromal cell that can differentiate into a variety of cell types, including, but not limited to osteoblasts (bone cells), chondrocytes (cartilage cells), myocytes (muscle cells) and adipocytes (fat cells). These cells are also known as “mesenchymal stem cells” due to their multipotency. This biologically important cell population is able to support haematopoiesis, can differentiate along mesenchymal and non-mesenchymal lineages in vitro, is capable of suppressing alloresponses and appears to be non-immunogenic.

The MSCs according to the invention is in some embodiments provided as a monogenic composition, therefore, such composition comprises cells of only one genetic background (or derived from one single donor subject). Preferred embodiments of the invention, however, then pertain to a cell preparation that comprises BMDC derived from multiple genetically distinct donor subjects. During the pooling method of the invention BMDC preparations may change the genetic background from monogenic to polygenic since at certain passages a pooling of multiple monogenic and distinct BDMCs are pooled into one cell culture. The number of donors providing the samples to be pooled for later BMDC isolation is preferably more than one preferably from at least 3, 4, 5, 6, 7, 8, 9 or 10 or more genetically distinct donor subjects. Preferably, BMDC according to the present invention are obtained from bone marrow samples, such as bone marrow mononuclear cell (BMNC) fractions, which are grown and pooled in passage p2. Preferably in context of the method of the invention only adherend cells are transferred into the new passage.

As used herein, the term “master cell bank” or “MCB” shall refer to a culture of cells that have been grown from a single donor sample, i.e. which are monogenic, and dispensed into storage containers, and stored preferably under cryopreservation conditions known to the skilled artisan. The cells may be suitable for later use in a production pooling method culture of the invention and to eventually harvest of the therapeutically clinical products. The term “working cell bank” or “WCB”, may either refer to a single donor bank of multiple monogenic cell cultures, or, and this is preferred, to an already pooled polygenic culture of cells that have been pooled from a single donor sample derived cultures, and optionally grown, i.e. which are polygenic, and dispensed into storage containers, and stored preferably under cryopreservation conditions known to the skilled artisan. The method of the invention in certain embodiments comprises that between the p0 and p1, a portion of cells is transferred into a storage receptacle and frozen to obtain a donor cell master cell bank, and wherein for continuing with p1, the cells of the storage receptacle are thawed for passaging into a cell culture receptacle for further cell culture in step (c).

In some embodiments the method of the invention is further comprising a step (h) passaging a portion of the cultured pooled cells in step (g) into a (separate) cell culture receptacle (p3). Preferably, cells obtained from the cells cultured cells in P3 can be used as clinical product suitable for a medical application.

The method of the invention is preferably for the isolation and generation of a pool of mesenchymal stromal cells (MSCs).

In context of the invention any subject, patient or donor is preferably a mammal and most preferably human.

The method of the invention may further comprise any additional step of testing the cells of any passage, cell bank or clinical product. Testing methods may include a testing for activity and cell number. A detailed testing is described herein below in the example sections. In any case, the testing may include the determination of the presence of one or more growth factors in the cells, or the cell medium. Such growth factors can be selected from the group brain-derived neurotrophic factor (BDNF), epidermal growth factor (EGF), Granulocyte-Colony Stimulating Factor, (G-CSF), hepatocyte growth factor (HGF), IL-1a, IL-6, LIF, osteopontin, Vascular Endothelial Growth Factor (VEGF)-A, fibroblast growth factor (FGF-2), transforming growth factor (TGF)-b, prostaglandin E (PG)E-2 and indoleamine 2,3-dioxygenase (IDO) after priming. Any one, or any combination of the foregoing may be tested for assessing cell or cell preparation quality.

In a second aspect, the invention pertains a working cell bank and or a clinical product produced with the method of the invention.

In a third aspect, the invention pertains to a preparation of clinical grade pooled mesenchymal stromal cells (MSC) produced with the method of the invention.

In a fourth aspect, the invention pertains to a preparation of clinical grade pooled mesenchymal stromal cells (MSC) for use in the treatment of a disease or condition in a subject.

In a fifth aspect, the invention pertains to a method for manufacturing a therapeutically active composition, such as a cell-free growth factor containing composition the method comprising performing a method according to the invention, or providing a working call bank and/or the clinical product, or the preparation, of any aspect of the invention, and subsequently culturing and/or expanding the MSC material in a cell culture, and harvest a composition into which the MSC material secreted one or more cell factors, to obtain the therapeutically active composition. Such compositions may preferably comprise any vesicles such as ECVs derived from the cell preparations of the invention, and/or growths factors as described.

It may be preferred that the therapeutically active composition is essentially cell free, such as a cell conditioned culture medium, optionally, subsequent to removing, for example by centrifuging, the cultured cells. Preferably, the medium is enrichted by the MSC of the invention.

Further preferred is that therapeutically active composition comprises one or more growth factors, preferably selected from brain-derived neurotrophic factor (BDNF), epidermal growth factor (EGF), Granulocyte-Colony Stimulating Factor, (G-CSF), hepatocyte growth factor (HGF), IL-1a, IL-6, LIF, osteopontin, Vascular Endothelial Growth Factor (VEGF)-A, fibroblast growth factor (FGF-2), transforming growth factor (TGF)-b, prostaglandin E (PG)E-2 and indoleamine 2,3-dioxygenase (IDO) after priming.

In a sixth aspect, the invention pertains to a therapeutically active composition, containing one or more growth factors, produced with the method of the invention.

In a seventh aspect, the invention provides a pharmaceutical composition, comprising a cell or cell-free material of any aspect of the invention, or obtained by, or obtainable by, any aspect of the invention.

The herein described methods and compositions are useful for an application in medicine, such as a therapeutic. Thus, for example, the mesenchymal stromal cells can be administered to an animal to treat blocked arteries, including those in the extremities, i.e., arms, legs, hands, and feet, as well as the neck or in various organs. For example, the mesenchymal stromal cells can be used to treat blocked arteries which supply the brain, thereby treating or preventing stroke. Also, the mesenchymal stromal cells can be used to treat blood vessels in embryonic and postnatal corneas and can be used to provide glomerular structuring. In another embodiment, the mesenchymal stromal cells can be employed in the treatment of wounds, both internal and external, as well as the treatment of dermal ulcers found in the feet, hands, legs or arms, including, but not limited to, dermal ulcers caused by diseases such as diabetes and sickle cell anemia. Moreover, the mesenchymal stromal cells or their cell-free derivatives can be administered intranasally. The MSCs and/or their cell-free derivatives, can be used as immunomodulation therapeutic for autoimmune diseases, alloimmune pathologies (including, but not restricted to, Graft-versus-Host Disease), as well in anti-tumor therapies. The uses described herein can both include the use of the cell material or any cell-free conditioned composition produced in accordance with the present invention.

Furthermore, because angiogenesis is involved in embryo implantation and placenta formation, the mesenchymal stromal cells can be employed to promote embryo implantation and prevent miscarriage. In addition, the mesenchymal stromal cells can be administered to an unborn subject, including humans, to promote the development of the vasculature in the unborn subject. In another embodiment, the mesenchymal stromal cells can be administered to a subject, born or unborn, in order to promote cartilage resorption and bone formation, as well as promote correct growth plate morphogenesis. The mesenchymal stromal cells can be genetically engineered with one or more (poly) nucleotides encoding a therapeutic agent, or further improving the mesenchymal stromal cells' function. The genetic engineering includes also genome editing of the mesenchymal stromal cells, e.g. with designer nucleases. The polynucleotides can be delivered to the mesenchymal stromal cells directly, or via an appropriate expression vehicle. Expression vehicles which can be employed to genetically engineer the mesenchymal stromal cells include, but are not limited to, retroviral vectors, adenoviral vectors, and adeno-associated virus vectors. The MSCs of the invention can for example be genetically engineered to overexpress TERT, and thereby to immortalize the cells. Also, the MSC preparation of the invention or the mesenchymal stromal cell can be for use in stem cell transplantation. Further provided is a use of the MSC preparation or MSCs of the invention in the production of bone replacement material.

The terms “of the [present] invention”, “in accordance with the invention”, “according to the invention” and the like, as used herein are intended to refer to all aspects and embodiments of the invention described and/or claimed herein.

As used herein, the term “comprising” is to be construed as encompassing both “including” and “consisting of”, both meanings being specifically intended, and hence individually disclosed embodiments in accordance with the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

It is to be understood that application of the teachings of the present invention to a specific problem or environment, and the inclusion of variations of the present invention or additional features thereto (such as further aspects and embodiments), will be within the capabilities of one having ordinary skill in the art in light of the teachings contained herein.

Unless context dictates otherwise, the descriptions and definitions of the features set out above are not limited to any particular aspect or embodiment of the invention and apply equally to all aspects and embodiments which are described.

All references, patents, and publications cited herein are hereby incorporated by reference in their entirety.

### BRIEF DESCRIPTION OF THE FIGURES

FIG. 1: (A) Pooling concept: hMSCs were isolated and expanded from bone marrow from six individual donors (passage p0). To initiate passage 1 expansion, single donor hMSCs were then thawed and either pooled at the onset of passage 1 (Pool 1) or expanded as single-donor hMSCs individually. These were then either pooled at the onset of passage 2 (Pool 2) or 3 (Pool 3), respectively. Pooled hMSCs at the end of passage 3 were cryopreserved, formulated as clinical product. (B) Master and Working Cell Bank concept: at the end of passage 0, single donor-derived hMSCs were cryopreserved as single donor MCBs. Single donor MSCs expanded in passage 1 were cryopreserved when harvested after passage 1 and served as WCBs. Pool 2 hMSCs, pooled at the onset of passage 2, were cryopreserved as working cell bank (WCB Pool 2) at the end of passage 2. Aliquots from this WCB were thawed and expanded one further passage to yield the potential clinical product end of passage 3. These cells were thawed and used for all experiments. Created with BioRender.com.

FIG. 2: Schematic of in vivo wound healing assay (A) Diabetic ZDF rats were wounded and treated topically with hMSCs (1×10⁶/cm² wound) in diluted fibrin glue. Untreated and cell-free fibrin glue-treated wounds served as control (created with BioRender.com). (B) Table representing animal allocation. Abbreviations: fresh=MSCs from max. 2 days of rescue-culture before administration to the wounds; cryo=MSCs thawed immediately before application, 1×=single application day 0, 3×=repeated application do, day 4 and day 8.

FIG. 3: shows All hMSC pools featured similar proliferation capacity, yet with Pool 2 hMSCs the highest number of extrapolated clinical doses could be achieved. All pools exerted similar functional characteristics. Calculation of (A) maximally achievable cell doses/manufacturing batch and (B) cell doses manufactured/batch (1×10⁶ hMSCs/cm² wound size) for hMSCs pooled at either passage 1 (Pool 1), passage 2 (Pool 2) or passage 3 (Pool 3). (C) Flow cytometry characterization of binary (absent or present) hMSCs markers. (D-H) Functional characterization of hMSC pools. Data are shown as normalized to Pool 2. (D) hMSCs-mediated inhibition of PHA-driven T cell proliferation. (E-H) Live cell imaging analyses of functional hMSC attributes: (E) proliferation; phase object confluence 96 h post-seeding; (F) scratch wound healing: relative wound density at 24 h post-wounding; (G) tube network formation: hMSCs were seeded as monolayer and fluorescently-labeled endothelial cells (HUVEC) were seeded on top. Tube length was assessed 48 h post-seeding and calculated as percent of human adipose stromal cell-mediated tube formation; (H) chemotactic migration to hPL-supplemented medium in bottom chamber. Counts of migrated cells in bottom wells were normalized to initial top-well values. Serum-free medium served as negative control. (E-H) Small letters indicate experimental replicates performed on different days by different operators. All data are shown as data from individual experimental replicates, indicating mean±SD.

FIG. 4: Pilot studies 1-3 to assess cryodamage, dose finding and systemic wound healing effects. (A) Representative images of wounded skin after single topical treatment with either hMSCs in fibrin glue, cell-free fibrin glue or untreated at do after wounding. (B) Wound area reduction after topical treatment with either untreated, cell-free fibrin glue and hMSCs thawed (cryo) or from rescue culture (fresh), (C) Single versus 3-times repeated hMSCs administration (1× versus 3× fresh, d0, 4 and 8). Quantification of wound areas relative to initial wound area was performed with Image J. Data are presented as min to max box-whisker plots denoting the median. (D, G) Comparison of wound size reduction of contralateral untreated wounds with lateral either hMSCs (D) or untreated wounds (E). Side-by side comparison of hMSCs fresh vs. contralateral untreated and untreated vs. contralateral untreated shown. *p≤0.05, **p≤0.01 as calculated using two-way ANOVA und Tukey multiple comparisons. Abbreviations: fresh=MSCs from max. 2 days of rescue-culture before administration to the wounds; cryo=MSCs thawed immediately before application, 1×=single application day 0, 3×=repeated application do, day 4 and day 8.

FIG. 5: hMSCs improve wound healing. (A) Wound size reduction after 3-times repeated topical treatment with either hMSCs (1×10⁶/cm²) in fibrin glue, cell-free fibrin glue or untreated at do, d4 and d8 after wounding. Quantification of wound areas relative to initial wound area was performed with Image J. *p≤0.05, ***p≤0.001, as calculated using two-way ANOVA and Tukey multiple comparisons. (B) Representative images of wounded skin after 3-times repeated topical treatment with either hMSCs in fibrin glue, cell-free fibrin glue or untreated depicting crust formation, especially in fibrin glue-treated wounds.

FIG. 6: hMSCs tend to increase lymphocyte infiltration, CD31+ vascularization and CD68- and CD163-positive macrophage infiltration, and to improve wound healing indices. Wound skin was harvested at d14 and histologically analyzed. (A) Frequencies of total cells, lymphocytes and fibroblasts within wounds analyzed by QuPath algorithm on HE stains. Values were normalized against the untreated contralateral site. (B) Representative images of CD31-positive structures and calculation relative to the total wound area outlined in yellow. (C-E) Frequencies of (C) CD31+, (D) CD68+ and (E) CD163+ cells were determined by immunohistochemical staining relative to the total wound area. (F-H) Histological wound healing indices: (F) Epithelial thickness index (ETI) calculated by comparing epithelial thickness in the uninjured skin and wounded area, (G) scar elevation index (SEI) calculated by comparing the dermis thickness in the uninjured skin and wounded area and (H) collagen density after Azan staining in the uninjured skin and wounded area. Quantification was done using QuPath algorithms. Data from individual wounds are shown. *p≤0.05, **p≤0.01, ***p≤0.001, ****p≤0.0001, as calculated using one-way ANOVA und Tukey-Test.

FIG. 7: hMSCs rapidly recruit macrophages, infiltrating wounds from the wound edges and basis. Immunohistochemical staining of (A, C, E) CD68- and (B, D, F) CD163-positive macrophages. (A, B) Quantification as described in FIG. 4. (C, D) Representative images are shown. (E, F) Wound margins are indicated in yellow, the histogreen-positive signal is highlighted and the histogreen-negative background signal reduced to visualize macrophage recruitment kinetics and routes.

FIG. 8: hMSCs are only transiently detectable in wounds. (A) Human Ku80-Histogreen staining in wound cross-sections. Left: Cross section of the entire wound. Right: Zoom on hKu80-positive cells. On day 1, wound edges with fibrin glue containing hMSCs located under the intact dermis are shown. Day 3 and 4 after hMSCs application, intact hMSCs were located in the fibrin glue top of the wound. Despite detection of human DNA in the wounds by dPCR on day 9, no hKu80 signal was detected in the histological sections. On day 11, very few hKu80-positive cells were found in the basal part of the wounds. (B) dPCR results of human DNA in rat wounds and organs. A value of ≥0.5 copies/μl was taken as positive result. (C) hKu80 expression in cryosections of analyzed organs: representative microphotographs are shown for samples where human cells were detected; no microphotographs are shown for negative samples.

FIG. 9: Summary of key findings. The present invention shows a novel clinical-scale manufacturing concept which is comprised of six single donor hMSCs master cell banks that are pooled to a working cell bank from which an extrapolated number of 70,000 clinical doses of 1×106 hMSCs/cm2 wound size can be manufactured within only three passages. Repeated topical hMSCs administration significantly accelerated the wound healing in a diabetic rat model by delivering a defined growth factor cargo at the specific stages of wound repair, namely inflammation, proliferation and remodeling. Specifically, the hMSCs mediated epidermal and dermal maturation and collagen formation, improved vascularization, and promoted cell infiltration, especially a dynamic recruitment of M2 macrophages.

FIG. 10 (Supplementary FIG. 1): All hMSC pools display similar adipogenic and osteogenic differentiation potential. hMSCs were seeded at a density of 1,000 cells/cm² and grown to subconfluency. Adipogenic differentiation was induced using the hMSC Adipogenic Differentiation Medium BulletKit (Lonza), or osteogenic differentiation was induced using osteogenic medium composed of α-MEM/10% FBS supplemented with 1 μM dexamethasone, 50 μM ascorbic acid, and 10 mM β-glycerolphosphate (Sigma-Aldrich). After 3 weeks under differentiation conditions, lipid vacuoles in adipogenic cultures were stained with oil red O and calcium deposits of osteogenic cultures with alizarin red S, respectively.

FIG. 11 (Supplementary FIG. 2): hMSCs migrate out of diluted fibrin glue. Instead of being applied to a wound, hMSCs were seeded in fibrin glue into a cell culture well plate and cultivated in medium supplemented with hPL versus serum-free medium as control. 4.5 hours post-seeding, hMSCs migrated from the glue attaining their typical fibroblast-like morphology. hMSCs in serum-free medium showed no migration out of the gel. (Axio Vert.A1, 5-fold magnification).

FIG. 12 (Supplementary FIG. 3): No systemic wound healing effect after topical hMSCs application. Rats were wounded and hMSCs applied topically in diluted fibrin glue. Comparison of wound size reduction of contralateral untreated wounds with lateral either (A) untreated, (B) hMSCs cryo single application, (C) fibrin glue single application, (D) single application hMSCs fresh, (E) repeated application of fibrin glue or (F) repeated application of hMSCs fresh d0, 4 and 8. Quantification of the wound area was performed with Image J. **p≤0.01, as calculated using two-way ANOVA und Sidak-Test.

### EXAMPLES

Certain aspects and embodiments of the invention will now be illustrated by way of example and with reference to the description, figures and tables set out herein. Such examples of the methods, uses and other aspects of the present invention are representative only, and should not be taken to limit the scope of the present invention to only such representative examples.

### Example 1: Closed System and Pooled hPL Allow Scale-Up Manufacture of Pooled hMSC Doses with Defined Trophic Factors Content

For scale-up and GMP-compatible manufacture, hMSCs were simultaneously cultured in standard Nunclon™ Delta flasks (175 cm² per flask) as well as in CELLSTACK™ with a larger culture surface (636 cm² per stack) enabling seeding, media exchange and harvest in the closed MC3 system. Growth kinetics and hMSC surface marker expression were identical (not shown). Production of clinical-scale doses was more feasible with the closed MC3 system due to optimized handling for media changes and passaging/harvest particularly reducing hands-on time in the cell culture.

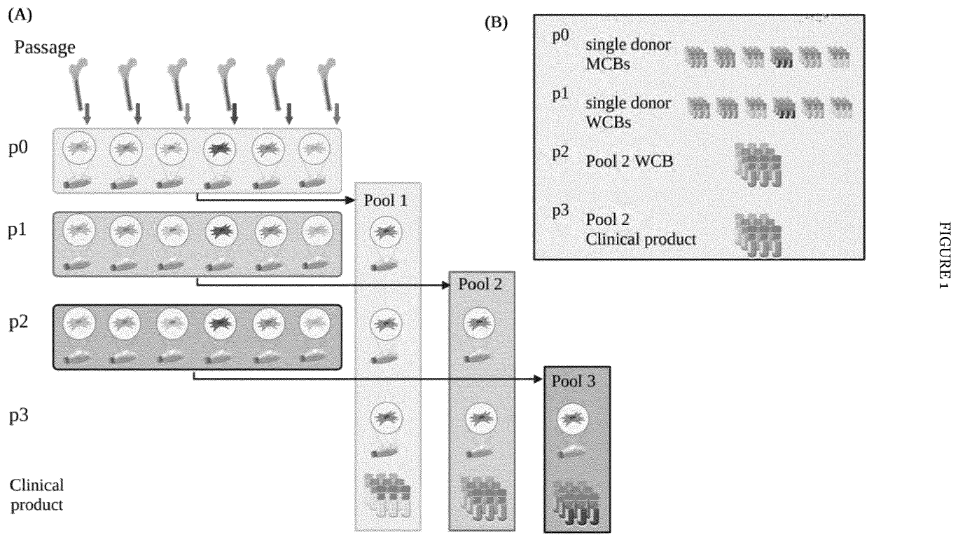

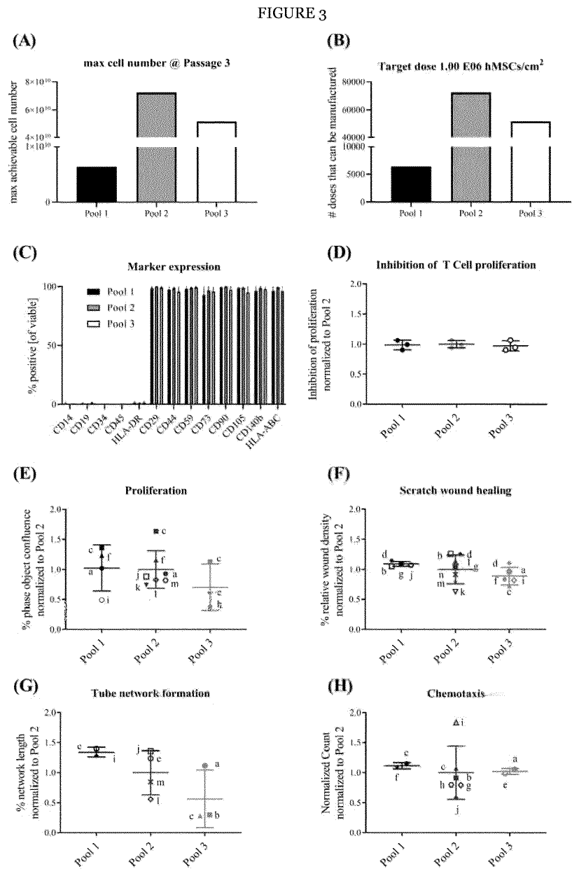

The extrapolated maximum of cell numbers that could be produced in line with highest number of target cell doses (~70,000 extrapolated doses) was achieved with Pool 2 hMSCs compared to Pool 1 (~6,000 extrapolated doses) and 3 (~50,000 extrapolated doses) hMSCs (FIG. 1A, B, FIG. 3A, B). For all hMSC pools, the expression of binary (absent or present) MSC markers was identical, consistent with guidelines set by the International Society for Cellular Therapy (FIG. 3C). The functional characterization of the hMSC pools proved similar regarding their adipogenic and osteogenic differentiation potential (Supplementary FIG. 1) and their immunomodulatory strength measured by their inhibition of PHA-driven T cell proliferation (FIG. 3D). Proliferation, scratch wound healing, vascular tube formation support and chemotactic migration assessed by live cell imaging showed also no differences between the hMSC pools (FIG. 3E-H). Yet, due to apparent day-to-day and operator-to-operator related variations in the latter assays, the need for better assay standardization became obvious.

Given that Pool 2 hMSCs achieved the highest calculated numbers of extrapolated clinical doses with similar characteristics compared to Pool 1 and 3 hMSCs, the inventors elected Pool 2 hMSCs for further preclinical evaluation.

The delivery of trophic factors is a key MoA of hMSCs (13). Therefore, the inventors quantitatively evaluated trophic factor candidates for wound healing. Of note, the inventors analyzed the hMSC lysates reflecting the actual clinical product, rather than mere cell culture supernatant collected during expansion. Specifically, the inventors detected BDNF, EGF, G-CSF, HGF, IL-1α, IL-6, LIF, osteopontin, VEGF-A, FGF-2, TGF-β, PGE-2 and inducible IDO-1 in the hMSCs and calculated their contents per applied hMSC dose (Table 1). GM-CSF, IL-1β, NGF-β, angiopoietin, IFN-γ, IL-2 and TNF-α were below the detection limit of the assay. These growth factors are active in different phases of wound healing.

TABLE 1
Trophic factors in Pool 2 hMSC lysate, calculated per hMSCs dose

| | pg/applied | | |
|---|---|---|---|
| | hMSCs | | |
| | dose | Wound healing function | References |
| Luminex | | | |
| BDNF | 3.06 | Acts proangiogenic | (58) |
| EGF | 0.58 | Induces migration, proliferation, plasticity of | (59, 60) |
| | | epithelial cells, fibroblast function, formation of | |
| | | granulation tissue | |
| G-CSF | 1.99 | Accelerates wound healing, promotes neutrophil | (61) |
| | | infiltration | |
| HGF | 122.68 | Induces migration, proliferation, and matrix | (59, 62) |
| | | metalloproteinase production of keratinocytes, | |
| | | acts proangiogenic | |
| IL-1α | 2.33 | Stimulates keratinocyte and fibroblast | (63) |
| | | proliferation, extracellular matrix remodeling, | |
| | | fibroblast chemotaxis, regulates the immune | |
| | | response | |
| IL-6 | 23.08 | Mitogenic for keratinocytes, promotes neutrophil | (59) |
| | | attraction | |
| LIF | 6.94 | Enhances proangiogenic potential of hMSCs | (64) |
| Osteopontin | 21.63 | Regulates ECM, myofibroblast differentiation | (58, 59) |
| VEGF-A | 174.43 | Acts proangiogenic | (58) |
| FGF2 | 2.69 | Acts proangiogenic, mitogenic for fibroblasts | (59) |
| | | and keratinocytes | |
| GM-CSF | below | Mitogenic for keratinocytes, induces migration | (58, 59) |
| | detection | and proliferation of endothelial cells, regulates | |
| | limit | macrophage polarization | |
| IL-1β | below | Acts proinflammatory | (58) |
| | detection | | |
| | limit | | |
| NGF-b | below | Stimulates nerve ingrowth | (59) |
| | detection | | |
| | limit | | |
| Angiopoietin | below | Induces vessel stabilization and remodeling | (59) |
| | detection | | |
| | limit | | |
| IFN-_{γ} | below | Modulates cell-mediated immunity, neutrophil | (65, 66) |
| | detection | inflammatory response, M1 polarization, can | |
| | limit | impair wound healing | |
| IL-2 | below | Attracts immune cells | (67, 68) |
| | detection | | |
| | limit | | |
| TNF-α | below | Proinflammatory, inhibits myofibroblast | (58) |
| | detection | differentiation | |
| | limit | | |
| ELISA | | | |
| TGF-b1 | 162.29 | Promotes chemoattraction, angiogenesis, M2 | (58, 59) |
| | | macrophage polarization, myofibroblast | |
| | | differentiation, mitogenic for fibroblasts, inhibits | |
| | | proliferation of keratinocytes, stimulates ECM | |
| | | proteins and integrin expression | |
| PGE2 | 26.57 | Induces anti-inflammatory responses, M2 | (69) |
| | | macrophage polarization, is proangiogenic, | |
| | | reduces pathological scar formation | |
| IDO-1 | 555.07 | Modulates immune responses | (70) |
| (after | | | |
| stimulation | | | |
| for 48 h | | | |
| with TNF-α, | | | |
| IL-1β, | | | |
| IFN-γ) | | | |

Abbreviations: BDNF—brain-derived neurotrophic factor, EGF—epidermal growth factor, G-CSF—granulocyte colony stimulating factor, HGF—hepatocyte growth factor, IL-1α—interleukin 1 alpha, IL-6—interleukin 6, LIF—leukemia inhibitory factor, VEGF-A—vascular endothelial growth factor A, FGF2—fibroblast growth factor 2, GM-CSF—granulocyte-macrophage colony-stimulating factor, IL-1β—interleukin 1 beta, NGF-β—nerve growth factor beta, IFN-γ—interferon gamma, IL-2—interleukin 2, TNF-α—tumor necrosis factor alpha, TGF-β1—transforming growth factor beta 1, PGE2—prostaglandin E2, IDO-1—indoleamine 2,3-dioxygenase.

Given that the media supplement influences the final trophic factors composition of the hMSC lysates (41), the inventors tested also the hPL batch used in this study (Table 2). Here, the inventors detected high concentrations of TGF-β 1, EGF, PDGF-AB and VEGF-A, mirrored by the relatively high amounts of TGF-β1 and VEGF-A in the hMSC dose (Table 1).

TABLE 2
Trophic factors content in hPL MultiPL′100i (batch 11219267DM),
including batch release test results. Experiments performed on
two different bags (#209 and 221) from the used MultiPL′100i
batch 11219267DM, means and standard deviations are shown.
hPL MultiPL′100i

| ELISA | pg/ml |
|---|---|
| bFGF | 85.43 ± 1.88 |
| IGF-1 | 95.34 ± 4.63 |
| TGF-b1 | 53647.50 ± 194.45 |
| EGF | 2287.7 ± 43.84 |
| PDGF-AB | 34257.00 ± 1032.38 |
| VEGF | 608.41 ± 13.30 |
| TNF-α ¹ | below detection limit |
| IFN-γ ¹ | below detection limit |
| batch release tests | |
| Endotoxins (LAL) | conform <1 UI/mL |
| Osmolality | 292 mOsmol/kg H2O |
| Turbidity | 140 NTU |
| pH | 7.5 |

¹ Experiments repeated twice with different controls and verified using ELISA kits from two different companies.
Abbreviations: bFGF—basic fibroblast growth factor, IGF—insulin-like growth factor 1, TGF-β1—transforming growth factor beta 1, EGF—epidermal growth factor, PDGF-AB, Platelet-derived growth factor AB, VEGF—vascular endothelial growth factor, TNF-α—tumor necrosis factor alpha, IFN-γ—interferon gamma, LAL—limulus amebocyte lysate, NTU—nephelometric turbidity unit.

### Example 2: HMSCs Migrate from the Fibrin Glue and Improve Skin Wound Healing in Diabetic Rats

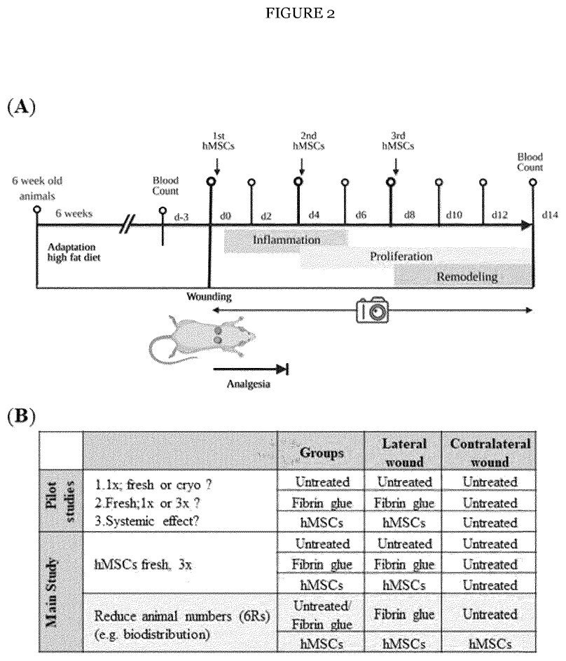

For cell application, the inventors used a protocol established by Yufit et al. using 1:10 diluted TISSEEL fibrin glue as cell carrier (9). In a pilot in vitro experiment, it was verified that hMSCs formulated in the 1:10 diluted fibrin glue were able to egress and migrate from the glue. The diluted fibrin glue needed about 7 minutes to polymerize to a gel. After 4.5 hours the hMSCs started to migrate from the glue into the culture vessel, and hMSC migration increased over time (Supplementary FIG. 2). Of note, no migration was induced in serum-free conditions indicating targeted migration of hMSCs.

For the in vivo evaluation of the wound healing potential of pooled hMSCs, three pilot studies were performed in preparation for the main study. In each study, two circular wounds of 8 mm diameter were set per one animal and either left untreated, treated with cell-free fibrin glue, or with hMSCs-formulated fibrin glue (FIG. 2A, B).

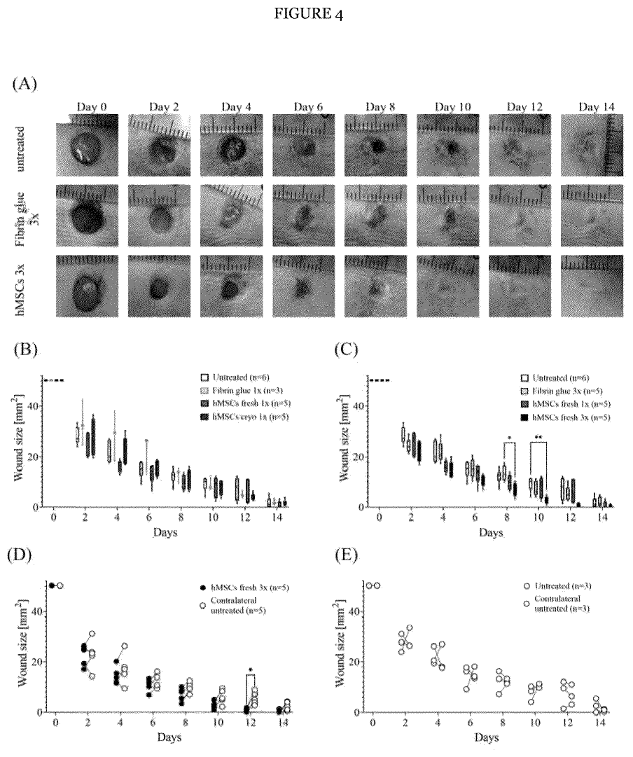

In pilot study 1, we evaluated an eventual cryodamage comparing just thawed hMSCs (cryo) with hMSCs from a rescue culture (fresh). From day 4 on, wounds treated with fresh hMSCs healed slightly better than hMSCs cryo (FIG. 4B). On day 12, a significantly smaller wound size was calculated in hMSC cryo-treated wounds compared to untreated wounds. Cell-free fibrin glue per se, compared to untreated wounds, promoted wound healing, but slightly delayed compared to hMSCs-treated wounds (d10 and d12, FIG. 4B). Based on these data, the inventors concluded that hMSCs show a slight cryodamage and favored the use of rescue-cultured fresh hMSCs for the subsequent experiments.

In pilot study 2, the inventors evaluated whether repeated application on days 0, 4 and 8 of fresh hMSC doses could further accelerate wound healing. Given that the hMSC therapeutic contained a large variety of growth factors, known to be active, and thus, being required, during the inflammation, proliferation and remodeling phase of wound healing, the inventors applied hMSCs at respective time points, do, d4 and d8 reflecting the different wound healing phases (FIG. 2A). Starting at day 4, wounds treated with hMSCs trended smaller than control-treated wounds, but from day 8 on, wounds treated 3 times with hMSCs were significantly smaller compared to controls (FIG. 4A, C).

### Example 3: Topically Applied hMSCs do not Exert Systemic Wound Healing Effects

Further, blood samples collected during the pilot studies were analyzed comparing white blood cells (WBCs), neutrophil, lymphocyte and platelet counts on day 0 and day 14. In the control settings, all blood cell counts appeared to be increased at day 14. Yet, WBCs and especially lymphocyte counts in 9 out of 15 hMSCs-treated animals were decreased compared to do. This effect was more pronounced after repeated hMSCs application (not shown). None of the control animals showed this trend.

Accordingly, the inventors asked in pilot study 3 whether hMSCs would exert systemic effects and could affect healing of the contralateral wound where no hMSCs were applied topically. Statistical analysis revealed that only the wounds that were topically treated with three hMSC doses at the different time points significantly improved healing. The contralateral untreated site showed comparable healing as untreated wounds (FIG. 4D, E, supplementary FIG. 3). These data suggest that topically applied hMSCs exert their therapeutic wound healing effects only locally.

Based on the results from these pilot studies and a power-based sample size calculation, the main study was designed (i) using fresh, rescue-cultured hMSCs, (ii) repeated application of hMSC doses on day 0, 4 and 8, and (iii) reduced numbers of control wounds according to the 6Rs principles, as systemic effects were excluded.

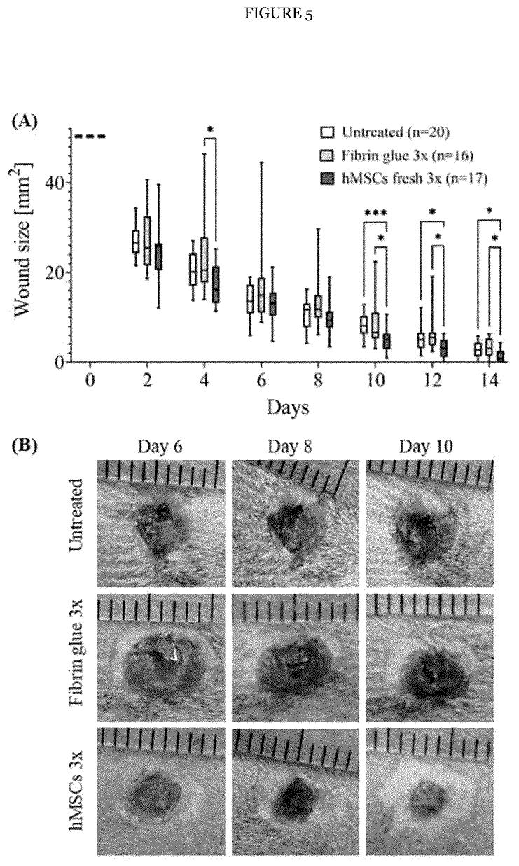

Results from the main study supported the significant wound healing effect of hMSCs. Wounds treated with three sequential hMSC doses were significantly smaller on days 10, 12 and 14 compared to both controls (FIG. 5A, B). Importantly, some wounds treated with hMSCs were already closed on day 12 after wound setting. In both control groups the first wounds were completely healed only at day 14. The inventors found that both control wounds, untreated and cell-free fibrin glue-treated, showed similar wound healing rates. In these series of experiments, the inventors observed extensive crust formation in fibrin glue-treated wounds, but not the hMSC-fibrin glue-treated wounds (FIG. 5).

The data from the pilot and the main studies documented that hMSCs significantly improved wound healing compared to both control groups. Yet, the initially observed trend of decreased circulating lymphocytes within peripheral blood after topical hMSCs application was not confirmed.

### Example 4: HMSCs Increase CD31-Positive Capillaries and Recruit CD68- and CD163-Positive Macrophages into Healing Wounds

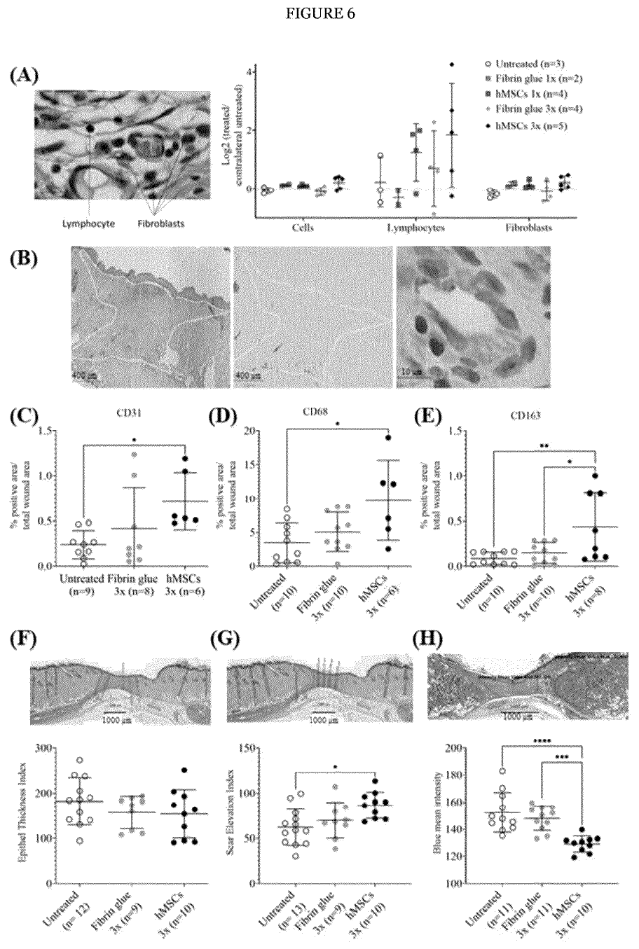

Having observed accelerated wound healing in hMSCs-treated wounds, histological analysis was performed. To gain insight into cell infiltration to the wounds, cells in total, lymphocytes and fibroblasts were identified based on their typical nuclear and cell phenotypic features (FIG. 6A). In the hMSCs-treated wounds more lymphocytes could be detected compared to untreated and fibrin glue-treated wounds, whereas fibroblast and total cell numbers seemed unaffected by hMSCs treatment (FIG. 6A).

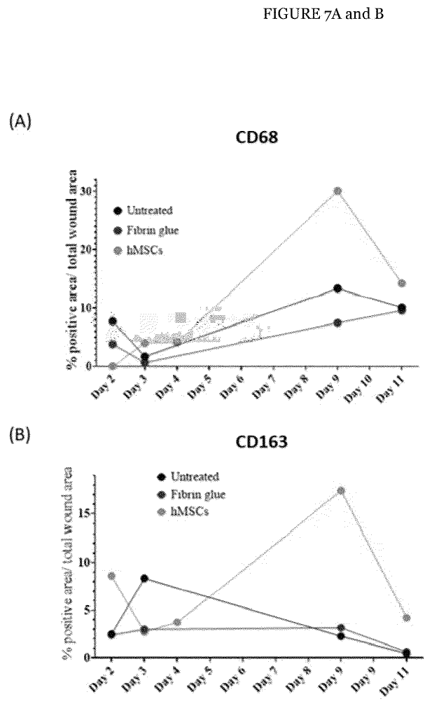

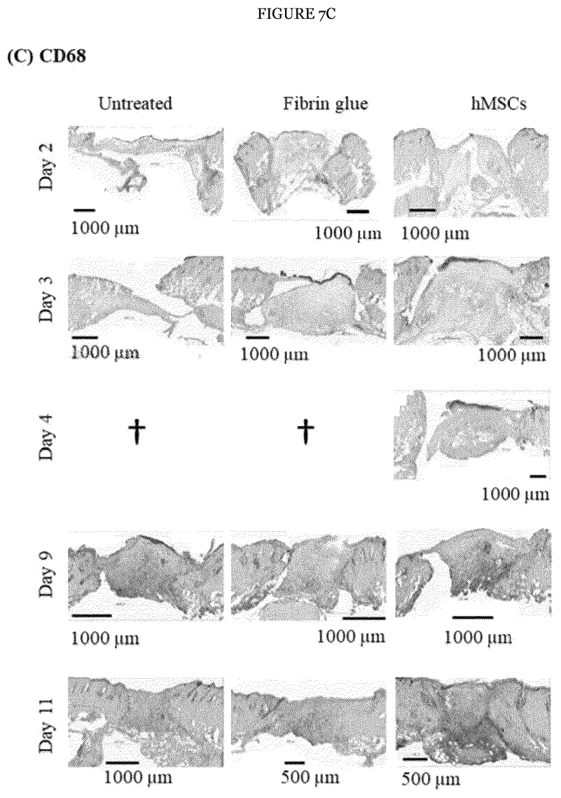

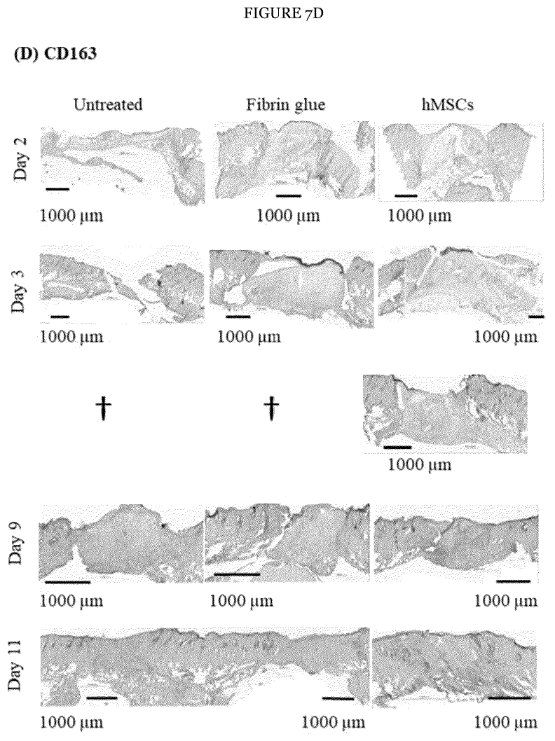

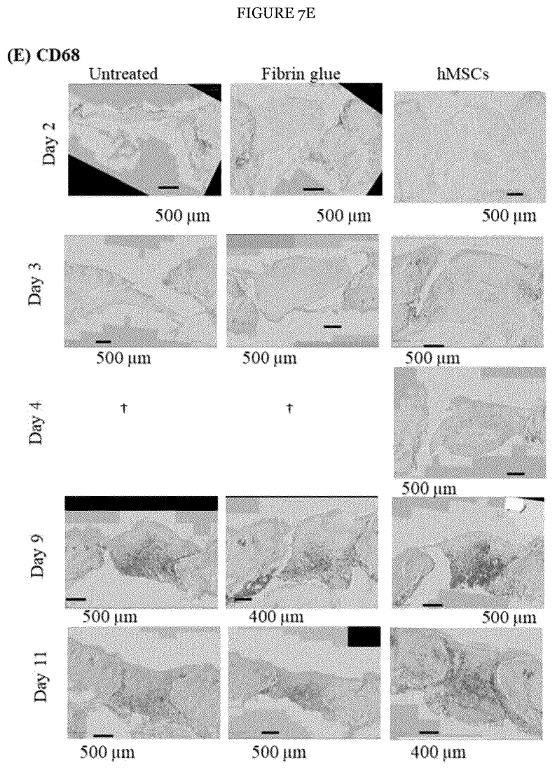

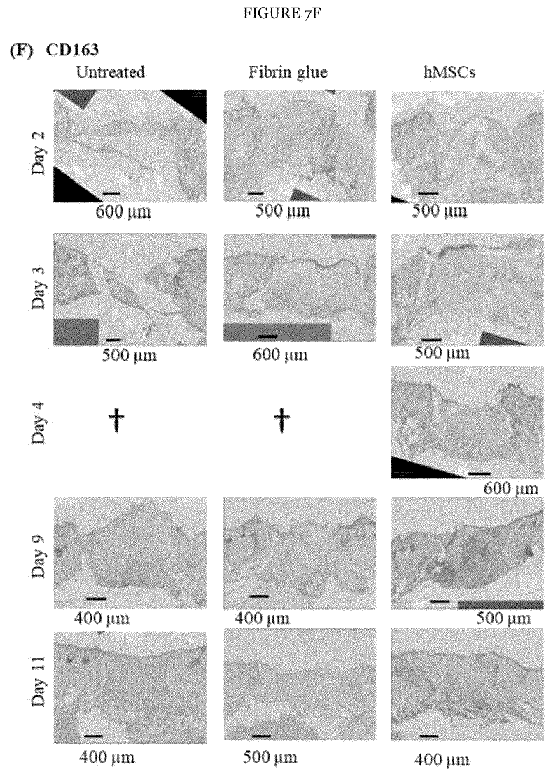

In a next step, immunohistochemical staining of CD31, indicative of tissue vascularization, CD68 as pan-macrophage marker and CD163 as marker for M2-subtype anti-inflammatory macrophages, activated in murine wound healing promoting to anti-inflammatory functions, extracellular matrix formation and angiogenesis (42), was performed. The inventors found an increase in CD31+ capillaries in the wounds repeatedly treated by hMSCs doses compared to untreated controls and fibrin glue-treated controls (FIG. 6B, C). The hMSCs-treated wounds showed also a higher proportion of CD68+ and CD163+ infiltrated macrophages, compared to both control groups (FIG. 6D, E). Detailed microscopic wound assessment at different time points after wound setting (part of the biodistribution study) revealed gradual infiltration of macrophages from the wound edges (d3), then the basal wound area (d9), eventually progressing to the apical wound tissue on d11 (FIG. 7). In the hMSCs-treated wounds, CD68+ cell infiltration extended more towards the apical layers than in both controls (FIG. 7C, E). This infiltration was accelerated in hMSCs-treated wounds at day 9 to then drop to similar levels as both controls on d11 (FIG. 7A). Starting at day 4, hMSCs promoted infiltration/differentiation of CD163-expressing macrophages, whereas both control-treated wounds revealed no increase in CD163-positive cells. Only in hMSC-treated wounds the CD163+ signal peaked at day 9 (FIG. 7B) with positive signals in the entire wound area extending to the apical layer (FIG. 7 D, F).

### Example 5: HMSCs Improve Epithelial Thickness, Reduce Scar Elevation and Increase Collagen Density in Healing Wounds

Having documented that hMSCs led to an increase in vascularization and induced CD68-, but also CD163-positive macrophage infiltration, the inventors aimed to gain more insights into the healing dynamics of the wounds. Here, a histology scoring system was used (39). First, an epithelial thickness index was calculated (FIG. 6F). Almost all wounds demonstrated hypertrophy of the epithelium, indicative for their healing stage (39). Without statistical significance, mean values suggested that the hMSCs-treated wounds showed the least epithelial thickness, followed by fibrin and then untreated wounds (FIG. 6F). Second, a scar elevation index was calculated. All wounds demonstrated hypoplasia of the dermis, yet hMSCs-treated wounds were already close to a normal state, significantly different to the untreated wounds, indicating an already better-developed wound compared to both controls (FIG. 6G). Third, collagen density was calculated based on intensity of blue Azan stain and compared to the respective non-wounded dermis. After migration into the wound, fibroblasts gradually produce ECM and collagen fibers. During wound healing, especially during proliferation stage, collagen accumulates in wounds, resulting in a darker blue Azan stain. Our results indicated a significantly higher collagen deposition and density in hMSCs-treated wounds on day 14 of the experiment compared to controls indicative of improved collagen deposition (FIG. 6H).

### Example 6: HMSCs are Only Transiently Detectable in Wounds

To assess the fate and biodistribution of topically applied hMSCs over time within the wounds and in distant organs, animals were sacrificed on day 1, 2, 3, 4, 9 and day 11, followed by histological as well as dPCR-based quantification of human cells.

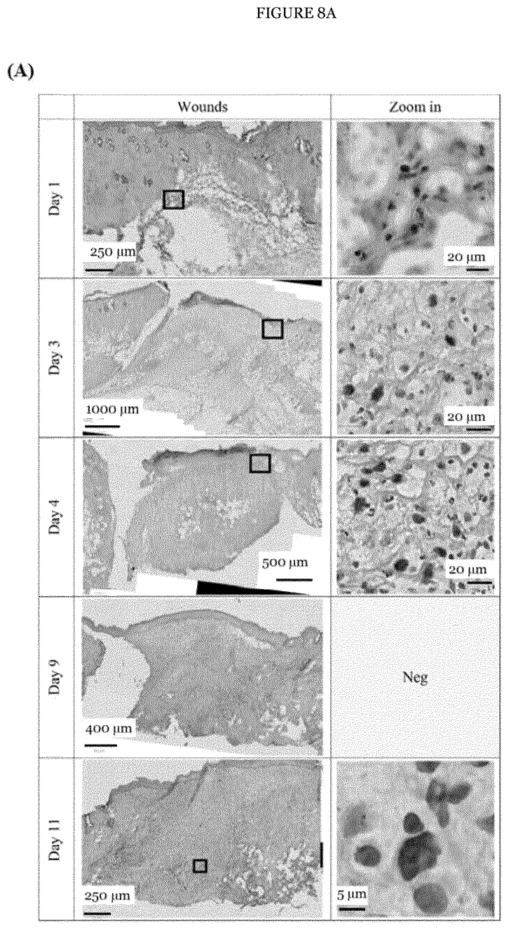

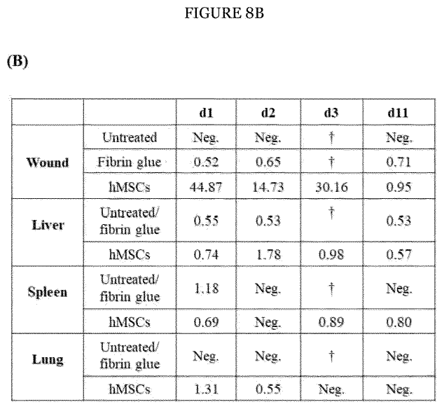

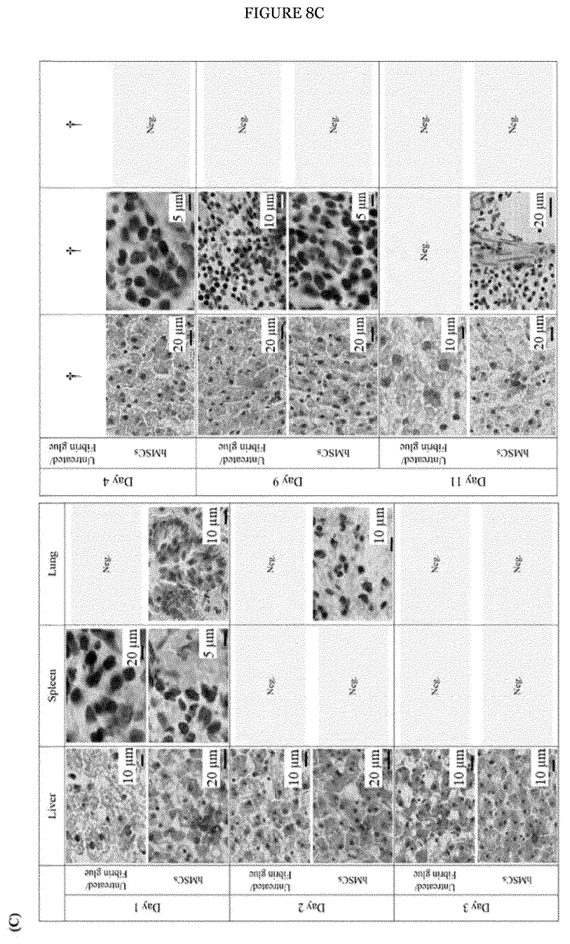

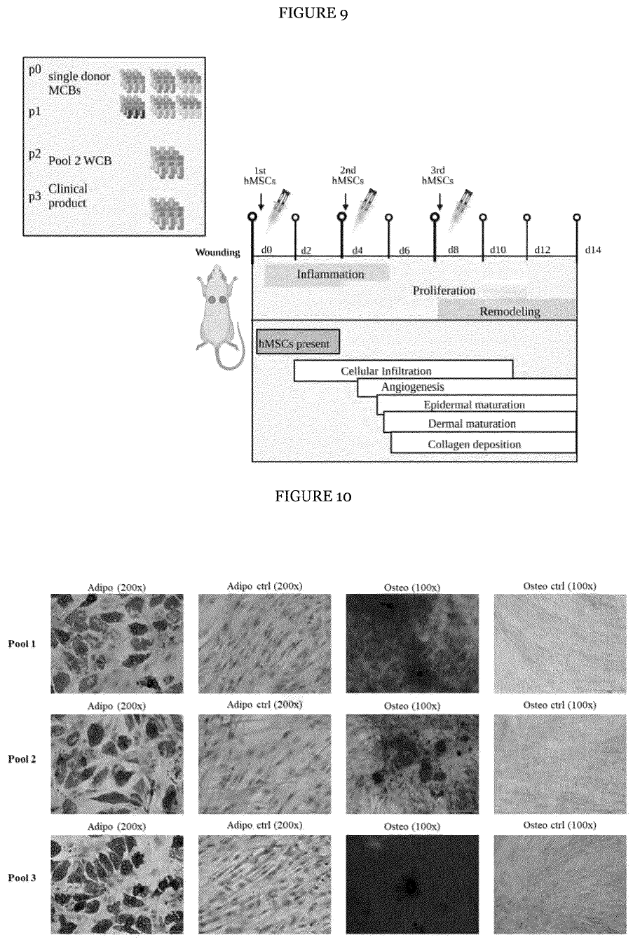

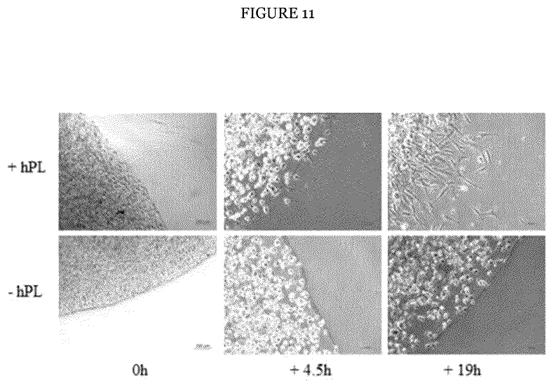

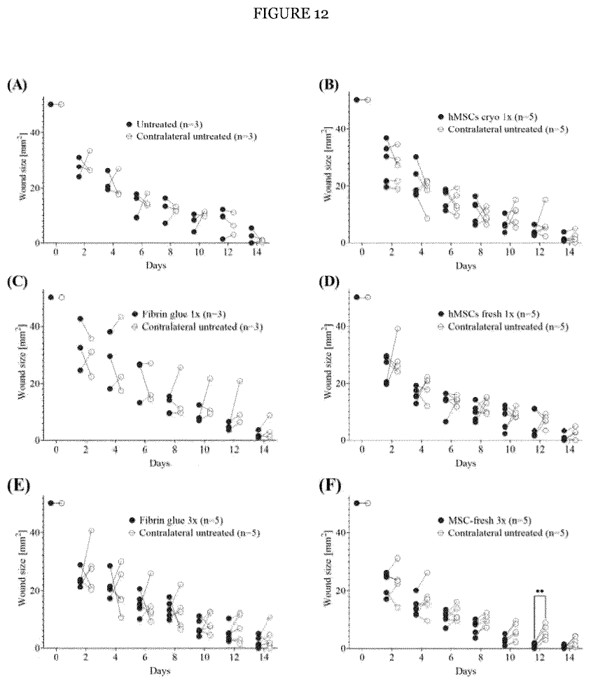

Staining the wound sections for human nuclear Ku80 expression (38), the inventors identified topically applied hMSCs on day 3 and 4 within the area of the fibrin glue interspersed within non-human tissue, yet hMSCs were undetectable at later time points (FIG. 8A).

Accordingly, the inventors confirmed the presence of human DNA in hMSCs-treated wounds. Levels decreased over time suggesting that the hMSCs were gradually eliminated from the wounds (FIG. 8B). Interestingly, traces of human DNA were also detected in cell-free fibrin glue-treated wounds, but never in untreated wounds, suggesting that the fibrin glue might contain low levels of human DNA. Human DNA was also detected in the livers of hMSCs-treated rats on day 1, 2, 4 and 11. The histological crosscheck revealed that the hKu80 signal was located in the cytoplasm, but not in the nucleus of the rat hepatocytes (FIG. 8C).

The inventors also detected traces of human DNA in livers of rats having one of their wounds treated with fibrin (FIG. 8B). These results were confirmed by histological analysis. Again, the hKu80 stain was cytoplasmic (FIG. 8C). It appears that not only hMSCs, but also the fibrin glue fragments, to a lesser extent, were transported from the wound site to the liver for phagocytosis by hepatocytes. Traces of human DNA were also found in the spleen of hMSCs-treated rats on days 1, 4 and 11 and in the spleens of animals with a fibrin glue-treated wound on day 1. Histological analysis confirmed this result as well (FIG. 8C). On day 1 and 2, dPCR detected the presence of human DNA in the lungs of hMSCs-treated rats. No human DNA was found in the lungs of fibrin glue-treated rats.

### BM-Derived HMSCS: Isolation, Cultivation and Characterization

Human BM-MNCs were obtained by puncturing the iliac crest of healthy BM donors (ethical vote #329/10, ethics committee, University Hospital Frankfurt am Main, Germany). The hBM-MNCs were seeded at a density of 100,000 cells/cm² in Nunclon™ Delta flasks in 93% alpha-MEM with Glutamin (Lonza, Cologne, Germany), 6% pooled virally inactivated human platelet lysate (hPL)²⁶ (MultiPL'100i, Macopharma, Tourcoing, France), 1% Penicillin/Streptomycin (Thermo Fisher Scientific, Darmstadt, Germany) and 2 IU Heparin (Ratiopharm GmbH, Ulm, Germany). After 24 h, the non-adherent cells were removed by rinsing with PBS (Thermo Fisher Scientific) and culture medium exchange, and hMSCs were grown from the adherent cell fraction (15). For scale-up and GMP-compatible manufacture, hMSCs were also cultured in CellStacks with a larger culture surface enabling seeding, media exchange and harvest in a closed system (MC3 system, Macopharma). After reaching subconfluence, hMSCs were split using TrypLE™ Select (Thermo Fisher Scientific) and seeded at a density of 1,000 cells/cm², or cryopreserved in 33% hPL, 5% DMSO (Sigma-Aldrich, Taufkirchen, Germany) in alpha-MEM as single donor-MCB. To level out individual differences, single donor hMSCs from six randomly chosen donors were then thawed and pooled at equal cell numbers (e.g. 6 times 1×10⁶ hMSCs), either at beginning of passage 1, 2 or 3 (Pool 1, 2 and 3, respectively). End of passage 3, hMSCs were cryopreserved as final (“clinical”) product. Pool 1 and 2 served as pooled WCBs (FIG. 1A). The hMSCs were verified to be mycoplasma—(Venor® GeM Classic, Minerva Biolabs GmbH, Berlin, Germany) and endotoxin-free (Endosafe® nexgen-PTS™, Charles River Laboratories, Freiburg, Germany).

Population doublings were calculated using the formula:

$$Population doublings ( PD ) = \frac{In ( \frac{harvest}{seed} )}{ln 2}$$

and maximum achievable cell number by

maximum achievable cell number=input*2^{population doubling}

Maximum achievable cell numbers at end of passage 3 and target cell dose equivalents (1×10⁶ hMSCs/cm² wound size) were extrapolated for Pools 1-3, respectively (32).

The hMSCs were characterized by a battery of in vitro test systems: First, marker expression (binary markers, either absent or present on hMSCs (33)) was assessed by flow cytometry (32). Second, adipogenic differentiation was induced using the hMSC Adipogenic Differentiation Medium BulletKit™ (Lonza, Basel, Switzerland) and osteogenic differentiation using osteogenic medium composed of alpha-MEM, 10% FBS supplemented with 1 μM dexamethasone, 50 μM ascorbic acid and 10 mM β-glycerolphosphate (Sigma-Aldrich), respectively. After three weeks of differentiation cells were lineage specifically stained: lipid vacuoles in adipogenic differentiation cultures were stained with Oil Red O, calcium deposits of osteogenic differentiated cells with Alizarin Red, respectively. Third, the hMSCs' capacity to inhibit T cell proliferation in vitro was assessed (32). Briefly, hMSCs were pre-seeded and CellTrace™ Violet (Thermo Fisher)-labeled pooled peripheral blood mononuclear cells (PBMNCs) were added, and further stimulated with phytohemagglutinin-L (PHA-L, 10 μg/mL, Sigma-Aldrich), or kept as non-stimulated controls. Proliferation of PBMNCs was assessed after 5 days using flow cytometry and hMSC-mediated inhibition was calculated. Fourth, live cell imaging using the Incucyte® Zoom device (Sartorius AG, Hertfordshire, United Kingdom) was performed and analyzed using Incucyte® analysis algorithms. First, hMSCs proliferation was assessed by seeding 200 hMSCs/cm² and monitoring the increase in cell confluence over time. The 96 h time point was chosen to compare Pools 1-3. Second, a scratch wound healing assay of hMSC monolayer was performed. In detail, hMSCs were seeded at 60,000 cells/cm² and incubated overnight. Then, wound scratches were applied using the 96-pin Incucyte® woundmaker tool. The wound closure (wound density at different time points relative to initial wound size) was calculated over time and values at 24 h used for comparison. Third, angiogenic tube network formation on hMSCs monolayers was assessed, as described previously (34). hMSCs were seeded at 60,000 cells/cm² and after 60 min 15,000 green fluorescent protein (GFP)+ human umbilical vein endothelial cells (HUVECs) were added. Human adipose-derived stromal cells (hASCs) served as positive control and were used for normalization of individual experiments. Network length (mm/mm²) was chosen as parameter for quantitative analysis. Fourth, chemotactic migration of hMSCs was assessed (35). Briefly, the insert plate of an Incucyte® ClearView 96 well plate was coated with fibronectin. Subsequently, 1,000 hMSCs were seeded and the plate was mated with the reservoir plate containing serum-free or hPL-containing medium. hMSCs migration was monitored for 48 h and analyzed as “count normalized to initial top value”.

Trophic factors of hMSCs lysate were quantified using Luminex and ELISA technologies (32). Briefly, 1-10×10⁶ hMSCs were harvested. hMSC pellets were lysed with ice-cold ProcartaPlex™ Cell Lysis Buffer, centrifuged at maximum speed and supernatant stored at −80 °C until assays were performed. Transforming growth factor beta 1 (TGF-β1) and Prostaglandin E2 (PGE2) were analyzed by ELISA (Biorbyt Ltd., Cambridge, UK and Cayman Chemical, Ann Arbor, MI, USA, resp.), Fibroblast growth factor 2 (FGF2) was analyzed by singleplex and all other trophic factors using a ProcartaPlex™ custom multiplex panel (Thermo Fisher Scientific).

hMSCs' IDO-1 production was stimulated by tumor necrosis factor-α (TNF-α), interleukin-1β (IL-1β), and interferon-γ (IFN-γ), each 20 ng/ml for 48 h. Subsequently, hMSCs were harvested and counted. Pellets were lysed (300 mM NaCl, 50 mM Tris, 2 mM MgCl₂, 0.05% NP40, 1× Protease/Phosphatase Inhibitor), centrifuged at maximum speed and supernatant stored at −80 °C until ELISA (Cloud-Clone Corp., Katy, TX, United States) was performed.

To assess the trophic factors content in the pooled hPL batch used in this study (MultiPL'100i; batch number 11219267DM), two different bags were tested by ELISA (Bio-techne; FGF (#SFB50), vascular endothelial growth factor-A (VEGF-A; #SVE00), epidermal growth factor (EGF; #SEG00), platelet-derived growth factor-AB (PDGF-AB) (#SHD00C), insulin-like growth factor-1 (IGF-1) (#SG100) and TGF-1 (#SB100B)).

### Wound Healing Model

Animal experiments were approved by the local ethics committee (G142-19, Regierungspräsidium Karlsruhe, Germany). Zucker diabetic rats were chosen as model of impaired wound healing (31). In total, 66 six weeks old male rats (ZDF (obese fa/fa), ZDF-Leprfa/Crl; Charles River Laboratories, Châtillon, France) were used. Upon arrival, rats were kept in groups of two and fed ad libitum with a special high-fat diet (Purina #5008, ssniff Spezialdiät GmbH, Soest, Germany) for 6 weeks to induce diabetes type II (FIG. 2A). Rats were weighed every 2 days and non-fasted blood glucose was measured once a week (Accu-Chek Aviva, Roche Diabetes Care, Mannheim, Germany). Animals were considered diabetic with a glucose level of 300 mg/dL, typically reached 3 weeks after initiating diet. Only diabetic rats were used for the wound healing experiments. Rats with blood glucose levels above 600 mg/dL were fed with the normal food until blood glucose levels dropped.

At 12 weeks of age, rats were anaesthetized with isoflurane (CP-Pharma 1 mL/mL, induction with 5% isoflurane plus 5 L/min oxygen and maintenance with 2-3% isoflurane plus 1 L/min oxygen) and 0.8 mL of blood were taken. For wound setting the rats were shaved on the back, the surgery field was disinfected and two wounds were set 1.5 cm behind the shoulder blades and 1.5 cm right and left from the spine with an 8 mm skin biopsy punch (WDT®, Garbsen, Germany). Only the skin was removed, the skeletal muscle fascia was left intact. Depending on the experimental setting, control animals were either left completely untreated or one wound was untreated and the other treated with cell-free fibrin glue. In other animals, one wound was treated with fibrin glue plus hMSCs, whereas the contralateral wound served as control, left untreated or treated with cell-free fibrin glue (FIG. 2B). Post-surgery 10 mL of physiological saline was injected subcutaneously to avoid dehydration and allow faster recovery after anaesthesia. To protect wounds from contamination or mutilation, a wound dressing was applied (Curapor®, Lohmann-Rauscher, Rengsdorf, Germany). This dressing was changed every other day. 200 mg/kg of metamizole sodium was used for analgesia (Novaminsulfon® solution for injection 500 mg/mL, Bela-pharm, Vechta, Germany) given for 4 days by subcutaneous injection.

For topical cell application, a commercial fibrin sealant syringe system was used (TISSEEL, Baxter Deutschland GmbH, Unterschleißheim, Germany). hMSCs were either thawed (cryo) or trypsinized after a short rescue culture (fresh; cells were thawed, cultured for up to two days to recover from eventual cryo-damage and to re-boot their metabolism), washed, counted and formulated at a density of 5×10⁵ viable hMSCs in 50 μL prediluted fibrinogen/aprotinin solution (final concentration 5 mg/mL) (9). Immediately before the application to the wounds, the cell suspension was drawn in one syringe of the duplojet device, while the other syringe contained prediluted thrombin solution (final concentration 25 mg/mL). Both components were combined using the TISSEEL duplojet system to formulate the fibrin glue. For each wound, 50 μL fibrinogen with hMSCs and 50 μL thrombin were then applied onto the wound, resulting in a dose of 1×10⁶ hMSCs/cm² wound. The glue was allowed to polymerize in the air for 7 minutes before applying the wound dressing.

To assess the capacity of the hMSCs to migrate out of the gel, an in vitro migration assay was performed. Briefly, fibrin glue with hMSCs was applied into a well of a 24-well plate. Subsequently, the hMSCs' migration towards hPL-supplemented culture medium as attractant, or serum-free medium as control, was evaluated microscopically.

Three pilot studies were performed, according to the 6R principles with each 3-5 animals. First, to pretest eventual cryopreservation damage the inventors compared freshly thawed (cryo) hMSCs against rescue culture post-thaw (fresh) hMSCs (28-30). Second, the inventors compared single dose injection (d0) against repeated hMSCs administration (days 0, 4 and 8) to apply hMSCs at the inflammation, proliferation and remodeling phase respectively (FIG. 2A). Of note, despite smaller wound sizes, the inventors applied the same cell dose as on do. Third, the inventors investigated eventual systemic effects of hMSCs. Here, single rats were allocated into one group where the contralateral site served as control, and the lateral site was treated with hMSCs and compared to a group of animals with only control-treated wounds.

With the results from the pilot study, a power analysis was performed to calculate the number of animals for the main study. Here, culture-adapted fresh hMSCs were applied repeatedly, but treatments of the two wounds were chosen randomly (in total, n=42 rats in the main study). To overcome potential breed-specific biases, experiments were performed in different experimental cohorts.

Furthermore, a biodistribution study was performed where animals were sacrificed on day 1, 2, 3, 4, 9 and 11 (each one animal per group). Here, both wounds served as either control (untreated/fibrin group) or were hMSCs-treated. Wound, liver, spleen and lungs were harvested, snap-frozen and then analyzed for the presence of human cells by immunohistochemical staining and digital PCR (dPCR).

For each animal, every other day upon wound dressing change, the wounds were scaled and photographed with a perpendicular angle. Wound area was measured using ImageJ (36). In addition, blood samples were taken after 14 days before the animals were sacrificed and a blood count performed (CELL-DYN Ruby, Abbott GmbH, Wiesbaden, Germany). First, the rats were fully anaesthetized with isoflurane and then 100 mg/kg ketamine and 5 mg/kg xylazine were injected intracardially. To avoid autolysis, wounds and organs were removed immediately. Wounds were cut in half to perform all analyses at the wounds center and were either paraformaldehyde (PFA)-fixed and paraffin-embedded or snap-frozen in Tissue-Tek® and cryomolds.

### Histological and Immunohistochemical Analysis

Standard hematoxylin-eosin (HE) and Azan staining was performed on 5 μm thick cuts after organ fixation in 4% PFA and paraffin embedding.

To investigate whether hMSCs promote host cell infiltration into the wound, artificial intelligence and QuPath algorithms (37) based on a random tree classifier were used for analyzing HE stains. First, the wound was defined as region of interest. Second, an automatic cell detection was run to determine the total cell count in this area. Using QuPath-based cell classification, fibroblasts and lymphocytes were discriminated based on nuclear stain (more homogenous and intense in lymphocytes than fibroblasts) and cellular morphology (round lymphocytes versus elongated fibroblasts). In addition, a “composite classifier” was used to improve the differentiation of lymphocytes characterized by their very pronounced circularity compared to fibroblasts.

Heidenhain's Azan trichrome stain was performed to assess collagen fiber deposition. Mean blue intensity was taken as measure of collagen density and dermis maturation. For this the “intensity mean value: blue” feature was used (Zeiss Zen 3.0 blue edition, Carl Zeiss Microscopy, Oberkochen, Germany). This tool calculates the average brightness (pixel value) of the selected region of interest. Dark blue colors reflecting high collagen density have lower pixel values than the lighter blue stains of wounds with fewer collagen fibers. Pixel values of the wound tissue were compared with those of the surrounding not injured dermis, with lower intensity equivalent to more collagen deposition in the granulation tissue.

$$Blue mean intensty in \% = \frac{wound intensity mean blue value * 100}{not injured dermis intensity mean blue value}$$

Immunohistochemical staining was performed to assess the degree of vascularization (CD31+ endothelial cells), immune cell infiltration (CD68+ and CD163+ macrophages) and presence of the transplanted hMSCs (human Ku80+ cells (38)). Cryosections (10 μm) were fixed in 4% PFA for 10 minutes. Nonspecific binding sites were blocked using 1% bovine serum albumin (BSA, PAN-Biotech, Aidenbach, Germany), 0.2% fish skin gelatin (Sigma-Aldrich) and 0.1% Triton X (Carl Roth, Karlsruhe, Germany) in Tris-Buffer saline. Antibodies were then added and incubated overnight (each 1:1,000 for mouse anti-rat monoclonal CD31 (Ab64543), Abcam, Cambridge, UK, rabbit anti-rat polyclonal CD68 (Ab125212) Abcam, mouse anti-rat monoclonal CD163 (MCA342GA) BioRad, Feldkirchen, Germany, and 1:250 rabbit anti-human monoclonal Ku80 (EPR3468), Abcam). After washing, endogenous peroxidase was blocked in 3% H₂O₂. Then, the secondary biotinylated antibody was added for 30 min (1:100 anti-mouse and anti-rabbit Ig, (RPN1001V, RPN1004V1) GE-healthcare, Solingen, Germany). Then 1% streptavidin peroxidase (GE-healthcare) was added. Histogreen was used as substrate chromogen (Linaris GmbH, Dossenheim, Germany). Nuclei were counterstained with Mayer's hematoxylin and sections mounted after dehydration in 99% ethanol, tissue clear and n-butyl acetate. Control slides were either left unstained to evaluate Histogreen background signal or stained with only the 2^{nd} antibody. Slides were scanned (Zeiss AXIO Scan.Z1) and analyzed using QuPath open software (36), creating a color filter to quantify histogreen-positive area in the entire wound previously defined as region of interest.

hKu80 staining in the organs was validated using Alexa Fluor 488- or Alexa Fluor 568-labeled secondary antibodies, (1:1,000; Life Technologies, Thermo Fisher Scientific) and TO-PRO-3 nuclear stain (Thermo Fisher Scientific) and assessed by confocal microscopy.

### Histology Scoring System

### Epidermal Thickness Index (ETI)

In 14 days old wounds, the average thickness of the wound epidermis was calculated for five locations and compared to the average thickness of the non-lesioned epidermis.

$$ETI = \frac{average epidermis thickness in wound area * 100}{average epidermis thickness in uninjured skin}$$

An ETI>105% is considered hypertrophic and mostly observed during the re-epithelialization phase and is an indicative of healing. A return of the epidermis thickness close to non-injured skin (95%<ETI<105%) is only observed after remodeling stage (39).

### Scar Elevation Index (SEI)

In 14 days old wounds, the average thickness of the dermis was calculated using five areas and compared to the average thickness of the unwounded dermis.

$$SEI = \frac{average dermis thickness in wound area * 100}{average dermis thickness in uninjured skin}$$

A hypertrophic dermis in the wound (SEI>105%) can reflect excessive collagen deposition and is therefore an indirect indicator of scar formation. A hypotrophic dermis with a SEI <95% is typically reported in early stages of healing wounds and reflects an underdeveloped dermis. A 95<SEI<105% characterizes a wound dermis whose thickness has returned to normal and is only observed in the final stage of healing (39).

### Chip-Based DPCR to Detect Residual Human Cells

To follow the fate of the topically applied hMSCs, wounds and organs were analyzed for human DNA using a sensitive dPCR method (40).

The dPCR assay was designed for detection of the single locus gene GAPDH in the rat and the human genome with specific primers and TaqMan™ probes with minor groove binding (MGB) modification at the 3′-end. For human GAPDH: forward primer, 5′-ccccacacacatgcacttacc-3′; reverse primer, 5′-cctagtcccagggctttgatt-3′; VIC-labeled probe, 5′-taggaaggacaggcaac-3′; for mouse/rat GAPDH: forward primer, 5′-gaatataaaattagatctctttggac-3′; reverse primer, 5′-gttgaatgcttggatgtacaacc-3′; FAM-labeled probe, 5′-taggaaggacaggcaac-3′. The human/rat GAPDH assay was prepared as 40× concentrated mixture containing 9 μmol of each primer and 5 μmol of each probe resulting in a final concentration of 225 nmol of each primer and 125 nmol of each probe.

The dPCR (QuantStudio® 3D; Thermo Fisher Scientific) was performed on chips with 20,000 reaction wells each with 755 μL volume. For each dPCR analysis 7.1 μL DNA was mixed with 0.375 μL 40× GAPDH assay and 7.5 μL dPCR Master Mix V2 containing ROX as reference dye (Thermo Fisher Scientific). The cycling program started with 10 min at 96 °C, followed by 40 cycles with 30 sec at 98 °C and 2 min at 52 °C. After cycling the dPCR chips were scanned for the FAM and VIC signals (QuantStudio® 3D Chip Reader; Thermo Fisher Scientific) and the data were analyzed using the QuantStudio 3D AnalysisSuite cloud software (https://apps.thermofisher.com/quantstudio3d). Based on the fluorescence signals and statistical correction using Poisson distribution the software enabled calculation of target copies per μL and target/total (%) values. For validation of the assay, human and rat genomic DNA was used pure and mixed at defined ratios (1:10, 1:20, 1:50). Human DNA was reliably detectable in the 1:50 mixture (detection limit 2%; approx. 4 copies/μL), whereas, pure rat DNA showed a background signal of 0.2% (approx. 0.4 copies/μL). 0.5 copies/μl were calculated as cut-off for positive signals.

Quantitative data are presented as means±standard deviation (SD) and were compared with analysis of variance (ANOVA) and post hoc tests as specified in the figures using GraphPad Prism (La Jolla, CA, USA). Values of p<0.05 were considered as statistically significant.

### REFERENCES

1. Otero-Vinas M, Falanga V. Mesenchymal Stem Cells in Chronic Wounds: The Spectrum from Basic to Advanced Therapy. Adv Wound Care (New Rochelle). 2016; 5 (4): 149-63.
2. Gianino E, Miller C, Gilmore J. Smart Wound Dressings for Diabetic Chronic Wounds. Bioengineering (Basel). 2018; 5 (3).
3. Galipeau J, Sensebe L. Mesenchymal Stromal Cells: Clinical Challenges and Therapeutic Opportunities. Cell Stem Cell. 2018; 22 (6): 824-33.
4. Krampera M, Le Blanc K. Mesenchymal stromal cells: Putative microenvironmental modulators become cell therapy. Cell Stem Cell. 2021; 28 (10): 1708-25.
5. Maxson S, Lopez E A, Yoo D, Danilkovitch-Miagkova A, Leroux M A. Concise review: role of mesenchymal stem cells in wound repair. Stem Cells Transl Med. 2012; 1 (2): 142-9.
6. Huang Y Z, Gou M, Da L C, Zhang W Q, Xie H Q. Mesenchymal Stem Cells for Chronic Wound Healing: Current Status of Preclinical and Clinical Studies. Tissue Eng Part B Rev. 2020; 26 (6): 555-70.
7. Marx C, Gardner S, Harman R M, Wagner B, Van de Walle G R. Mesenchymal stromal cell-secreted CCL2 promotes antibacterial defense mechanisms through increased antimicrobial peptide expression in keratinocytes. Stem Cells Transl Med. 2021; 10 (12): 1666-79.
8. Falanga V, Iwamoto S, Chartier M, Yufit T, Butmarc J, Kouttab N, et al. Autologous bone marrow-derived cultured mesenchymal stem cells delivered in a fibrin spray accelerate healing in murine and human cutaneous wounds. Tissue Eng. 2007; 13 (6): 1299-312.
9 Yufit T, Carson P, Falanga V. Topical delivery of cultured stem cells to human non-healing wounds: GMP facility development in an academic setting and FDA requirements for an IND and human testing. Curr Drug Deliv. 2014; 11 (5): 572-81.
10. Kerstan A, Dieter K, Niebergall-Roth E, Dachtler A K, Kraft K, Stucker M, et al. Allogeneic ABCB5(+) mesenchymal stem cells for treatment-refractory chronic venous ulcers: a phase I/IIa clinical trial. JID Innov. 2022; 2 (1).
11. Kiritsi D, Dieter K, Niebergall-Roth E, Fluhr S, Daniele C, Esterlechner J, et al. Clinical trial of ABCB5+ mesenchymal stem cells for recessive dystrophic epidermolysis bullosa. JCI Insight. 2021; 6 (22).
12. Armstrong J P K, Keane T J, Roques A C, Patrick P S, Mooney C M, Kuan W L, et al. A blueprint for translational regenerative medicine. Sci Transl Med. 2020; 12 (572).
13. Bieback K, Kuci S, Schafer R. Production and quality testing of multipotent mesenchymal stromal cell therapeutics for clinical use. Transfusion. 2019; 59 (6): 2164-73.
14. Galipeau J. The mesenchymal stromal cells dilemma—does a negative phase III trial of random donor mesenchymal stromal cells in steroid-resistant graft-versus-host disease represent a death knell or a bump in the road? Cytotherapy. 2013; 15 (1): 2-8.
15. Siegel G, Kluba T, Hermanutz-Klein U, Bieback K, Northoff H, Schafer R. Phenotype, donor age and gender affect function of human bone marrow-derived mesenchymal stromal cells. BMC Med. 2013; 11:146.
16. Kuci Z, Bonig H, Kreyenberg H, Bunos M, Jauch A, Janssen J W, et al. Mesenchymal stromal cells from pooled mononuclear cells of multiple bone marrow donors as rescue therapy in pediatric severe steroid-refractory graft-versus-host disease: a multicenter survey. Haematologica. 2016; 101 (8): 985-94.
17. Bader P, Kuci Z, Bakhtiar S, Basu O, Bug G, Dennis M, et al. Effective treatment of steroid and therapy-refractory acute graft-versus-host disease with a novel mesenchymal stromal cell product (MSC-FFM). Bone Marrow Transplant. 2018; 53 (7): 852-62.
18. Bonig H, Kuci Z, Kuci S, Bakhtiar S, Basu O, Bug G, et al. Children and Adults with Refractory Acute Graft-versus-Host Disease Respond to Treatment with the Mesenchymal Stromal Cell Preparation “MSC-FFM”-Outcome Report of 92 Patients. Cells. 2019; 8 (12).
19. Gupta P K, Krishna M, Chullikana A, Desai S, Murugesan R, Dutta S, et al. Administration of Adult Human Bone Marrow-Derived, Cultured, Pooled, Allogeneic Mesenchymal Stromal Cells in Critical Limb Ischemia Due to Buerger's Disease: Phase II Study Report Suggests Clinical Efficacy. Stem Cells Transl Med. 2017; 6 (3): 689-99.
20. Thej C, Balasubramanian S, Rengasamy M, Walvekar A, Swamynathan P, Raj S S, et al. Human bone marrow-derived, pooled, allogeneic mesenchymal stromal cells manufactured from multiple donors at different times show comparable biological functions in vitro, and in vivo to repair limb ischemia. Stem Cell Res Ther. 2021; 12 (1): 279.
21. Bieback K, Hecker A, Schlechter T, Hofmann I, Brousos N, Redmer T, et al. Replicative aging and differentiation potential of human adipose tissue-derived mesenchymal stromal cells expanded in pooled human or fetal bovine serum. Cytotherapy. 2012; 14 (5): 570-83.
22. Blumel J, Schwantes A, Baylis S A, Stuhler A. Strategies toward virus and prion safe human platelet lysates. Transfusion. 2020; 60 (1): 219-20.
23. Burnouf T, Strunk D, Koh MB, Schallmoser K. Human platelet lysate: Replacing fetal bovine serum as a gold standard for human cell propagation? Biomaterials. 2016; 76:371-87.
24. Astori G, Amati E, Bambi F, Bernardi M, Chieregato K, Schafer R, et al. Platelet lysate as a substitute for animal serum for the ex-vivo expansion of mesenchymal stem/stromal cells: present and future. Stem Cell Res Ther. 2016; 7 (1): 93.
25. Viau S, Eap S, Chabrand L, Lagrange A, Delorme B. Viral inactivation of human platelet lysate by gamma irradiation preserves its optimal efficiency in the expansion of human bone marrow mesenchymal stromal cells. Transfusion. 2019; 59 (3): 1069-79.
26. The European Agency for the Evaluation of Medicinal Products. Human Medicines Evaluation Unit. Note for guidance on virus validation studies: The design, contribution and interpretation of studies validating the inactivation and removal of viruses. CPMP/BWP/268/95 1996.
27. Francois M, Copland I B, Yuan S, Romieu-Mourez R, Waller E K, Galipeau J. Cryopreserved mesenchymal stromal cells display impaired immunosuppressive properties as a result of heat-shock response and impaired interferon-gamma licensing. Cytotherapy. 2012; 14 (2): 147-52.
28. Chinnadurai R, Copland I B, Garcia M A, Petersen C T, Lewis C N, Waller E K, et al. Cryopreserved Mesenchymal Stromal Cells Are Susceptible to T-Cell Mediated Apoptosis Which Is Partly Rescued by IFNgamma Licensing. Stem Cells. 2016; 34 (9): 2429-42.
29. Chinnadurai R, Garcia MA, Sakurai Y, Lam WA, Kirk AD, Galipeau J, et al. Actin cytoskeletal disruption following cryopreservation alters the biodistribution of human mesenchymal stromal cells in vivo. Stem Cell Reports. 2014; 3 (1): 60-72.
30 Moll G, Geissler S, Catar R, Ignatowicz L, Hoogduijn M J, Strunk D, et al. Cryopreserved or Fresh Mesenchymal Stromal Cells: Only a Matter of Taste or Key to Unleash the Full Clinical Potential of MSC Therapy? Adv Exp Med Biol. 2016; 951:77-98.
31. Slavkovsky R, Kohlerova R, Tkacova V, Jiroutova A, Tahmazoglu B, Velebny V, et al. Zucker diabetic fatty rat: a new model of impaired cutaneous wound repair with type II diabetes mellitus and obesity. Wound Repair Regen. 2011; 19 (4): 515-25.
32. Spohn G, Witte A S, Kretschmer A, Seifried E, Schafer R. More Human BM-MSC With Similar Subpopulation Composition and Functional Characteristics Can Be Produced With a GMP-Compatible Fabric Filter System Compared to Density Gradient Technique. Front Cell Dev Biol. 2021; 9:638798.
33. Dominici M, Le Blanc K, Mueller I, Slaper-Cortenbach I, Marini F, Krause D, et al. Minimal criteria for defining multipotent mesenchymal stromal cells. The International Society for Cellular Therapy position statement. Cytotherapy. 2006; 8 (4): 315-7.
34. Kremer H, Gebauer J, Elvers-Hornung S, Uhlig S, Hammes H P, Beltramo E, et al. Pro-angiogenic Activity Discriminates Human Adipose-Derived Stromal Cells From Retinal Pericytes: Considerations for Cell-Based Therapy of Diabetic Retinopathy. Front Cell Dev Biol. 2020; 8:387.
35. Wuhrer A, Uhlig S, Tuschy B, Berlit S, Sperk E, Bieback K, et al. Wound Fluid from Breast Cancer Patients Undergoing Intraoperative Radiotherapy Exhibits an Altered Cytokine Profile and Impairs Mesenchymal Stromal Cell Function. Cancers (Basel). 2021; 13 (9).
36. Schneider C A, Rasband W S, Eliceiri K W. NIH Image to ImageJ: 25 years of image analysis. Nat Methods. 2012; 9 (7): 671-5.
37. Bankhead P, Loughrey M B, Fernandez J A, Dombrowski Y, McArt D G, Dunne P D, et al. QuPath: Open source software for digital pathology image analysis. Sci Rep. 2017; 7 (1): 16878.
38. Rigon M, Horner S J, Straka T, Bieback K, Gretz N, Hafner M, et al. Effects of ASC Application on Endplate Regeneration Upon Glycerol-Induced Muscle Damage. Front Mol Neurosci. 2020; 13:107.
39. van de Vyver M, Boodhoo K, Frazier T, Hamel K, Kopcewicz M, Levi B, et al. Histology Scoring System for Murine Cutaneous Wounds. Stem Cells Dev. 2021; 30 (23): 1141-52.
40. Eryilmaz M, Muller D, Rink G, Kluter H, Bugert P. Introduction of Noninvasive Prenatal Testing for Blood Group and Platelet Antigens from Cell-Free Plasma DNA Using Digital PCR. Transfus Med Hemother. 2020; 47 (4): 292-301.
41. Bieback K, Hecker A, Kocaomer A, Lannert H, Schallmoser K, Strunk D, et al. Human alternatives to fetal bovine serum for the expansion of mesenchymal stromal cells from bone marrow. Stem Cells. 2009; 27 (9): 2331-41.
42. Krzyszczyk P, Schloss R, Palmer A, Berthiaume F. The Role of Macrophages in Acute and Chronic Wound Healing and Interventions to Promote Pro-wound Healing Phenotypes. Front Physiol. 2018; 9:419.
43. Martin I, Galipeau J, Kessler C, Le Blanc K, Dazzi F. Challenges for mesenchymal stromal cell therapies. Sci Transl Med. 2019; 11 (480).
44. Schrezenmeier H, Seifried E. Buffy-coat-derived pooled platelet concentrates and apheresis platelet concentrates: which product type should be preferred? Vox Sang. 2010; 99 (1): 1-15.
45. Kerstan A, Niebergall-Roth E, Esterlechner J, Schroder H M, Gasser M, Waaga-Gasser A M, et al. Ex vivo-expanded highly pure ABCB5(+) mesenchymal stromal cells as Good Manufacturing Practice-compliant autologous advanced therapy medicinal product for clinical use: process validation and first in-human data. Cytotherapy. 2021; 23 (2): 165-75.
46. Chen L, Tredget E E, Wu P Y, Wu Y. Paracrine factors of mesenchymal stem cells recruit macrophages and endothelial lineage cells and enhance wound healing. PLOS One. 2008; 3 (4): e1886.
47. Fontaine M J, Shih H, Schafer R, Pittenger M F. Unraveling the Mesenchymal Stromal Cells' Paracrine Immunomodulatory Effects. Transfus Med Rev. 2016; 30 (1): 37-43.
48. Wu Y, Chen L, Scott P G, Tredget E E. Mesenchymal stem cells enhance wound healing through differentiation and angiogenesis. Stem Cells. 2007; 25 (10): 2648-59.
49. Song J Y, Lim J W, Kim H, Morio T, Kim K H. Oxidative stress induces nuclear loss of DNA repair proteins Ku70 and Ku80 and apoptosis in pancreatic acinar AR42J cells. J Biol Chem. 2003; 278 (38): 36676-87.
50. Galleu A, Riffo-Vasquez Y, Trento C, Lomas C, Dolcetti L, Cheung T S, et al. Apoptosis in mesenchymal stromal cells induces in vivo recipient-mediated immunomodulation. Sci Transl Med. 2017; 9 (416).
51. Ankrum J A, Ong J F, Karp J M. Mesenchymal stem cells: immune evasive, not immune privileged. Nat Biotechnol. 2014; 32 (3): 252-60.
52. Avivar-Valderas A, Martin-Martin C, Ramirez C, Del Rio B, Menta R, Mancheno-Corvo P, et al. Dissecting Allo-Sensitization After Local Administration of Human Allogeneic Adipose Mesenchymal Stem Cells in Perianal Fistulas of Crohn's Disease Patients. Front Immunol. 2019; 10:1244.
53. Ardanaz N, Vazquez F J, Romero A, Remacha A R, Barrachina L, Sanz A, et al. Inflammatory response to the administration of mesenchymal stem cells in an equine experimental model: effect of autologous, and single and repeat doses of pooled allogeneic cells in healthy joints. BMC Vet Res. 2016; 12:65.
54. Ballikaya S, Sadeghi S, Niebergall-Roth E, Nimtz L, Frindert J, Norrick A, et al. Process data of allogeneic ex vivo-expanded ABCB5(+) mesenchymal stromal cells for human use: off-the-shelf GMP-manufactured donor-independent ATMP. Stem Cell Res Ther. 2020; 11 (1): 482.
55. Viswanathan S, Keating A, Deans R, Hematti P, Prockop D, Stroncek D F, et al. Soliciting strategies for developing cell-based reference materials to advance mesenchymal stromal cell research and clinical translation. Stem Cells Dev. 2014; 23 (11): 1157-67.
56. Stroncek D F, Jin P, McKenna D H, Takanashi M, Fontaine M J, Pati S, et al. Human Mesenchymal Stromal Cell (MSC) Characteristics Vary Among Laboratories When Manufactured From the Same Source Material: A Report by the Cellular Therapy Team of the Biomedical Excellence for Safer Transfusion (BEST) Collaborative. Front Cell Dev Biol. 2020; 8:458.
57. Chaires-Rosas C P, Ambriz X, Montesinos J J, Hernandez-Tellez B, Pinon-Zarate G, Herrera-Enriquez M, et al. Differential adhesion and fibrinolytic activity of mesenchymal stem cells from human bone marrow, placenta, and Wharton's jelly cultured in a fibrin hydrogel. J Tissue Eng. 2019; 10:2041731419840622.
58. Rodrigues M, Kosaric N, Bonham C A, Gurtner G C. Wound Healing: A Cellular Perspective. Physiol Rev. 2019; 99 (1): 665-706.
59. The inventors rner S, Grose R. Regulation of wound healing by growth factors and cytokines. Physiol Rev. 2003; 83 (3): 835-70.
60. Hardwicke J, Schmaljohann D, Boyce D, Thomas D. Epidermal growth factor therapy and wound healing-past, present and future. Surg-J R Coll Surg E. 2008; 6 (3): 172-7.
61. Jimi S, Sato K, Kimura M, Suzumiya J, Hara S, De Francesco F, et al. G-CSF Administration Accelerates Cutaneous Wound Healing Accompanied With Increased Pro-Hyp Production In db/db Mice. Clinical Research in Dermatology: Open Access. 2017; 4 (2): 1-9.
62. Conway K, Ruge F, Price P, Harding K G, Jiang W G. Hepatocyte growth factor regulation: an integral part of why wounds become chronic. Wound Repair Regen. 2007; 15 (5): 683-92.
63. Robertson F M, Pellegrini A E, Ross M S, Oberyszyn A S, Boros L G, Bijur G N, et al. Interleukin-1alpha gene expression during wound healing. Wound Repair Regen. 1995; 3 (4): 473-84.
64 Santos G C, Silva D N, Fortuna V, Silveira B M, Orge I D, de Santana T A, et al. Leukemia Inhibitory Factor (LIF) Overexpression Increases the Angiogenic Potential of Bone Marrow Mesenchymal Stem/Stromal Cells. Front Cell Dev Biol. 2020; 8:778.
65. Kanno E, Tanno H, Masaki A, Sasaki A, Sato N, Goto M, et al. Defect of Interferon gamma Leads to Impaired Wound Healing through Prolonged Neutrophilic Inflammatory Response and Enhanced MMP-2 Activation. International Journal of Molecular Sciences. 2019; 20 (22).
66. Miles R H, Paxton T P, Zacheis D, Dries D J, Gamelli R L. Systemic Administration of Interferon-Gamma Impairs Wound-Healing. J Surg Res. 1994; 56 (3): 288-94.
67. Seraphim P M, Leal E C, Moura J, Goncalves P, Goncalves J P, Carvalho E. Lack of lymphocytes impairs macrophage polarization and angiogenesis in diabetic wound healing. Life Sci. 2020; 254.
68. Doersch K M, DelloStritto D J, Newell-Rogers M K. The contribution of interleukin-2 to effective wound healing. Exp Biol Med. 2017; 242 (4): 384-96.
69 Cheng H, Huang H, Guo Z, Chang Y, Li Z. Role of prostaglandin E2 in tissue repair and regeneration. Theranostics. 2021; 11 (18): 8836-54.
70. Ito H, Ando T, Ogiso H, Arioka Y, Saito K, Seishima M. Inhibition of indoleamine 2,3-dioxygenase activity accelerates skin wound healing. Biomaterials. 2015; 53:221-8.

1. A method for the production of a donor cell pool of expandable cells derived from bone marrow samples of multiple genetically non-identical bone marrow donors, the method comprising the steps of:
(a) Providing two or more bone marrow donor samples of genetically non-identical (or distinct) bone marrow donors,
(b) Separately passaging bone marrow derived cells from each bone marrow donor sample of (a) into a (separate) cell culture receptacle, such as a culture flask or dish, or bioreactor (p0);
(c) Culturing the bone marrow derived cells of each donor separately;
(d) Separately passaging a portion of each culture of bone marrow derived cells from (c) into a new cell culture receptacle, such as a culture flaks or dish (p1);
(e) Culturing the bone marrow derived cells of each donor separately;
(f) Passaging and pooling (combining) a portion of each culture of bone marrow derived cells from (e) into one single cell culture receptacle obtain the donor cell pool (p2),

2. The method of claim 1, further comprising a step (f′) subsequent to (f) freezing and storing the donor cell pool as a working cell bank.

3. The method of claim 1, further comprising a step (g) culturing the donor cell pool, either directly from step (f), or after thawing a cell sample of the working cell bank of claim 2.

4. The method of claim 1, further comprising a step (h) passaging a portion of the cultured pooled cells in step (g) into a (separate) cell culture receptacle (p3).

5. The method of claim 4, obtaining the cells cultured cells as clinical product suitable for a medical application.

6. The method of claim 1, wherein passaging involves transferring a portion of cultured cells from a cell culture receptacle into a new receptacle comprising new cell culture medium.

7. The method of claim 1, wherein the donors are human donors.

8. The method of claim 1, wherein between the p0 and p1, a portion of cells is transferred into a storage receptacle and frozen to obtain a donor cell master cell bank, and wherein for continuing with p1, the cells of the storage receptacle are thawed for passaging into a cell culture receptacle for further cell culture in step (c).

9. A working cell bank and or a clinical product produced by, or obtainable by, a method according to claim 1.

10. A preparation of clinical grade pooled mesenchymal stromal cells (MSCs), obtainable by a method of claim 1.

11. A method of treatment of a disease or condition in a subject, comprising a step of administering to the subject in need of the treatment a preparation of clinical grade pooled mesenchymal stromal cells (MSCs) obtained by or obtainable by the method of claim 1.

12. A method for manufacturing a therapeutically active composition, the method comprising performing a method according to claim 1, or providing a working call bank and/or the clinical product obtained by, or obtainable by, a method according to claim 1, or providing a preparation of clinical grade pooled mesenchymal stromal cells (MSCs), obtainable by a method of claim 1, culturing and/or expanding the MSC material in a cell culture, and harvest a composition into which the MSC material secreted one or more cell factors, to obtain the therapeutically active composition.

13. The method of claim 12, wherein the therapeutically active composition comprises a one or more growth factors and/or other bioreactive molecule, preferably selected from brain-derived neurotrophic factor (BDNF), epidermal growth factor (EGF), Granulocyte-Colony Stimulating Factor, (G-CSF), hepatocyte growth factor (HGF), IL-1a, IL-6, LIF, osteopontin, Vascular Endothelial Growth Factor (VEGF)-A, fibroblast growth factor (FGF-2), transforming growth factor (TGF)-b, prostaglandin E (PG)E-2 and indoleamine 2,3-dioxygenase (IDO) after priming; and/or wherein the therapeutically active composition comprises MSC derived extra cellular vesicles (ECVs).

14. A therapeutically active composition, such as a cell-free composition, obtained or obtainable by the method of claim 12.

15. The therapeutically active composition of claim 14, which is a pharmaceutical composition, comprising further one or more pharmaceutically acceptable carrier and/or excipient.
